(19) United States
(12) Patent Application Publication
(10) Pub. No.: US 2025/0062037 A1
(43) Pub. Date: Feb. 20, 2025

(54) SYSTEMS AND DEVICES FOR COUPLING METABOLOMICS DATA WITH DIGITAL MONITORS FOR PRECISION HEALTH

(71) Applicants: DUKE UNIVERSITY, Durham, NC (US); The Governors of the University of Alberta, Edmonton (CA)

(72) Inventors: Rima Kaddurah-Daouk, Durham, NC (US); David Wishart, Edmonton (CA); Dorsa Yahya Rayat, Edmonton (CA)

(21) Appl. No.: 18/837,460

(22) PCT Filed: Jan. 25, 2023
(86) PCT No.: PCT/CA2023/050092

§ 371 Date: Aug. 9, 2024

Related U.S. Application Data
(60) Provisional application No. 63/303,156, filed on Jan. 26, 2022.
(60) Provisional application No. 63/302,652, filed on Jan. 25, 2022.

(51) Int. Cl. G16H 50/70; A61B 5/00; G16B 20/00; G16H 10/60; G16H 20/10; G16H 20/30; G16H 20/60; G16H 20/90

(52) U.S. Cl. CPC G16H 50/70; A61B 5/4866; G16B 20/00; G16H 10/60; G16H 20/10; G16H 20/30; G16H 20/60; G16H 20/90

(57) ABSTRACT

The present disclosure describes, in part, devices, systems, and methods for connecting metabolomic measurements to big data being generated by sensors captured in databases to enable monitoring of metabolic health at molecular level to map disruptions in biochemical processes in each individual that can inform about basis of disease and ways to correct for such defects tailored for each individual.

### CROSS-REFERENCE TO RELATED APPLICATIONS

This application claims priority to U.S. Provisional Patent Application No. 63/302,652, filed on Jan. 25, 2022; and U.S. Provisional Patent Application No. 63/303,156, filed on Jan. 26, 2022; each of which is incorporated by reference herein in its entirety.

### FIELD

This disclosure relates generally to systems, devices and methods for coupling or connecting metabolomics data with data from digital monitors for precision health and/or wellbeing.

### BACKGROUND

Recent developments in high-throughput omics screening technologies and their broad application in biomedical research, the use of medical records and the development of sensors (digital health) that enable continuous collection of data on health is all creating highly informative data about the health of individuals and in real time. This combined with use of machine-learning methods and AI technologies provides great promise for optimizing health for each individual. However, there remains a need for coupling or connecting metabolomics measurements to big data being generated by sensors captured in databases to enable monitoring of metabolic health at the molecular level to map disruptions in biochemical processes in an individual that can inform about health, development of disease, disease subtypes, response to treatment, lifestyle interventions and their molecular impact on health. This information adds in powerful ways to a signal from digital recording and is actionable as it provides readout for monitoring health and interventions that can sustain health. It enables a precision medicine and precision health approach.

### SUMMARY

The Summary is provided to introduce a selection of concepts that are further described below in the Detailed Description. This Summary is not intended to identify key or essential features of the claimed subject matter, nor is it intended to be used as an aid in limiting the scope of the claimed subject matter.

The present disclosure is based, in part, on the discovery by the inventors of devices, systems, and methods for connecting metabolomics measurements to big data being generated by sensors captured in databases, to enable monitoring of metabolic health at a molecular level to map disruptions in biochemical processes in a subject that can inform about basis of health, disease and ways to correct for such defects, that is tailored for the subject. The use of sensors combined with molecular signatures is a powerful approach for providing precision health and precision medicine approaches, thereby tailoring best treatment for each individual.

In some embodiments, the devices, systems, and methods provided herein provide for monitoring of health and wellness of a subject. In some embodiments, the devices, systems, and methods provided herein provide for instructing the subject on ways to adjust their lifestyle to improve their health and wellness. In some embodiments, the devices, systems, and methods provided herein may relate the outcome of a subject's efforts to improve their health and wellness to their metabolomic data.

According to one aspect, the present disclosure provides for a device capable of measuring one or more biomarkers as provided herein. Other aspects of the present disclosure provide for a system for measuring a plurality of biomarkers provided herein. For any individual, the plurality of biomarkers makes up the individual's metabolomic profile.

Yet other aspects of the present disclosure provide for a method of monitoring the health and wellness of a subject, the method comprising, consisting of, or consisting essentially of measuring one or more biomarkers to create a metabolomic profile, analyzing the biomarkers, optionally comparing the results with that of a larger biomarker database (e.g., metabolomic data) compiled from a plurality of users, and optionally, providing an output for instructing the subject on an intervention to improve their health and wellness based on the results.

In some embodiments, metabolic measurements or signatures are derived by providing a biological sample from a subject who is also using digital recording for health-related data, and where a biological sample such as a blood sample is analyzed on a metabolomics platform, which may include GC/LC mass spectrometry or NMR. Examples of biological samples include, but are not limited to, urine, saliva, breath/respiration (e.g., for use in a breathalyzer), other body fluids such as plasma, tears, bile, spinal fluid, mucus, lymph, serum, blood, and the like, skin or tissue, biopsies, among others. According to one embodiment, one or more metabolites are (i) extracted from the sample; (ii) analyzed; (iii) optionally connected to digital recording for health such as smart watch and (iv) compared to a database (e.g., a metabolomics database) which are then compiled in an output that is provided to the subject (or user, such as a medical professional). The output may provide, for example, information relating to the health and wellness of the subject, instructions to the subject on an intervention, which may comprise an adjustment to improve their health and wellness (e.g., changes in diet, and/or exercise, use of supplements and/or medications, and the like).

In some embodiments, the biological sample comprises a drop of blood. For example, in such embodiments, the drop of blood may be placed on a piece of filter paper as is being done for newborn screening, or through a capillary device where a biological sample, such as a drop of blood, is absorbed by a cotton swab as in mitra device or similar approaches or by providing blood samples in clinical setting in a conventional manner, or through home collection using a device such as a Tasso™ device. Any method is suitable which enables continuous monitoring of chemicals by extracting metabolites from the the blood sample and analyzing for chemicals using methods such as LC and GC mass spectrometry or NMR.

In some embodiments, additional genetic data (such as those available through commercial providers such as 23&Me™ and Ancestry™) and/or gut microbiome data, are further measured and incorporated into or combined with the metabolic profile and, optionally, in the metabolomic data, wherein any metabolic defects are also mapped to a possible genetic variation or gut bacterial enzymatic reactions adding net influences of genome and gut microbiome on metabolome. Similarly, effects of exposome can be connected to the metabolomic data and evaluated.

In another aspect, disclosed is a method of improving the health or wellness of a subject. A first step is to create a master database (referred to herein as the MetabosensorDB) by collecting continuous digital health data and metabolomic data for a population of individuals over an extended time period where at least one perturbation, and preferably multiple perturbations (diet, lifestyle, etc.) are applied. The population of individuals is preferably diversely representative. Once the MetabosensorDB is created, standard multivariate statistical methods are used to group members of the population into health stratification groups such as metabotypes (metabolic health phenotypes or MHPs) and digital health phenotypes (DHPs) in MetaboSensorDB. The method then comprises the steps of:
(a) Collecting digital health and metabolic data for a subject over a shorter time period;
(b) Comparing the subject's digital health phenotype (DHP) and metabolic health phenotype (MHP) to see which health stratification group in the MetaboSensorDB the subject matches with;
(c) Apply a perturbation to the subject, which perturbation is associated with a desired health or wellness outcome for individuals in the health stratification group matched by the subject.

In some embodiments, the subject may continue to collect digital health data and/or metabolomic data and determine the effect of the perturbation on the DHP and/or MHP of the subject, which is added to the MetabosensorDB. In some embodiments, if the subject experiences a change in DHP and/or MHP which is different than the matched health stratification group, the subject may be moved to another health stratification group, or a subgroup may be identified. The subgroup may consist of the subject alone.

In some embodiments, the MetaboSensorDB also comprises one or both of genomic profile data and microbiome profile data from the population of individuals. The subject may then also collect and compare such genomic profile data and/or microbiome profile data in the matching step to find a matching health stratification group.

In some embodiments, the shorter time period is a single 24 hour period, or any period shorter or longer than a single 24 hour period.

In another aspect, disclosed is a system for improving the health or wellness of a subject; comprising:
(a) a master database (MetabosensorDB) comprising digital health data and metabolomic data collected for a population of individuals over an extended time period where at least one perturbation is applied, and where standard multivariate statistical methods are used to group members of the population into a plurality of health stratification groups;
(b) at least one device for collecting digital health data from a subject;
(c) a metabolomic profile derived from a biological sample from the subject;
(d) a computer operatively connected to the at least one device and configured to:
i. receive the subject digital health data and metabolomic profile, and compare to the plurality of health stratification groups to match the subject to a health stratification group; and
   ii. identify at least one perturbation which is predicted to improve the subject's health or wellness; and
iii. communicate the predicted at least one perturbation to the subject.

Another aspect of the present disclosure provides all that is described and illustrated herein.

### BRIEF DESCRIPTION OF THE DRAWINGS

The accompanying Figures and Examples are provided by way of illustration and not by way of limitation. The foregoing aspects and other features of the disclosure are explained in the following description, taken in connection with the accompanying example figures (also “Fig.”) relating to one or more embodiments, in which.

FIG. 1 is a schematic showing the MetaboSensorDB and a subject's digital health data, metabolomic profile, microbiome profile and genomic profile.

FIG. 2 is a schematic flowchart of an exemplary method disclosed herein.

FIG. 3 shows pictures showing sleep disruption mood changes and metabolism in accordance with one embodiment of the present disclosure.

FIG. 4 is a picture, image, and schematic showing the system and methods of using a sensor to collect information for coupling metabolomics data with digital monitors for precision health in accordance with one embodiment of the present disclosure.

FIGS. 5A and 5B are schematics showing system and methods of using a sensor (e.g., smart watch) to collect information for coupling metabolomics data with digital monitors for precision health in accordance with one embodiment of the present disclosure.

FIG. 6 shows a metabolomics PCA scores plot of the heart rate digital health status groupings (DHSGs).

FIG. 7 shows a metabolomics PLS-DA scores plot of heart rate DHSGs.

FIG. 8 shows a metabolomics PCA scores plot of the mental performance DHSGs.

FIG. 9 shows a metabolomics PLS-DA scores plot of the mental performance DHSGs.

FIG. 10 shows a metabolomics PCA scores plot of blood glucose DHSGs.

FIG. 11 shows a metabolomics PLS-DA scores plot of blood glucose DHSGs.

FIG. 12 shows a metabolomics PCA scores plot of sleep quality DHSGs.

FIG. 13 shows a metabolomics PLS-DA scores plot of the sleep quality DHSGs.

FIG. 14 shows a VIP plot of the most important metabolites and metabolite ratios that are associated with heart rate.

FIG. 15 shows a VIP plot of the most important metabolites and metabolite ratios that are associated with mental performance.

FIG. 16 shows a VIP plot of the most important metabolites and metabolite ratios that are associated with blood glucose levels.

FIG. 17 shows a VIP plot of the most important metabolites and metabolite ratios that are associated with sleep quality.

FIG. 18 shows a graph of blood pressure measurements from a test subject.

FIG. 19 shows heart rate data from the test subject.

FIG. 20 shows blood glucose data from the test subject during a fast food diet.

FIG. 21 shows blood glucose data from the test subject during a Mediterranean style diet.

FIG. 22 shows blood glucose data from the test subject during a ketogenic diet.

FIG. 23 shows body weight data from the test subject.

FIGS. 24, 25A and 25B shows gut microbiome data from the test subject.

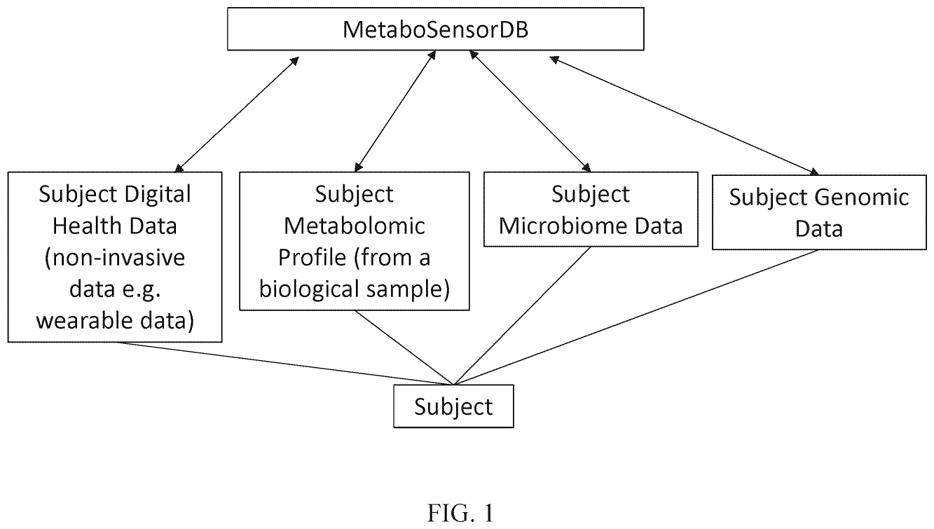

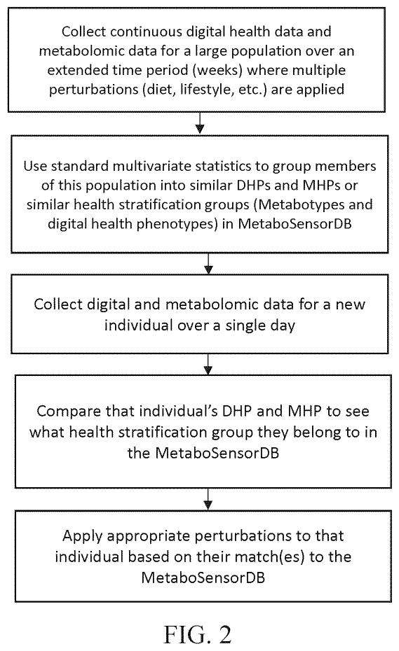

### DETAILED DESCRIPTION

For the purposes of promoting an understanding of the principles of the present disclosure, reference will now be made to preferred embodiments and specific language will be used to describe the same. It will nevertheless be understood that no limitation of the scope of the disclosure is thereby intended, such alteration and further modifications of the disclosure as illustrated herein, being contemplated as would normally occur to one skilled in the art to which the disclosure relates.

Articles “a” and “an” are used herein to refer to one or to more than one (i.e. at least one) of the grammatical object of the article. By way of example, “an element” means at least one element and can include more than one element.

“About” is used to provide flexibility to a numerical range endpoint by providing that a given value may be “slightly above” or “slightly below” the endpoint without affecting the desired result. The variation may be 10% above or below the stated value, or may be indicative of a level of imprecision in measurement.

The use herein of the terms “including,” “comprising,” or “having,” and variations thereof, is meant to encompass the elements listed thereafter and equivalents thereof as well as additional elements. As used herein, “and/or” refers to and encompasses any and all possible combinations of one or more of the associated listed items, as well as the lack of combinations where interpreted in the alternative (“or”).

As used herein, the transitional phrase “consisting essentially of” (and grammatical variants) is to be interpreted as encompassing the recited materials or steps “and those that do not materially affect the basic and novel characteristic(s)” of the claimed invention. Thus, the term “consisting essentially of” as used herein should not be interpreted as equivalent to “comprising.”

Moreover, the present disclosure also contemplates that in some embodiments, any feature or combination of features set forth herein can be excluded or omitted. To illustrate, if the specification states that a system comprises components A, B, and C, it is specifically intended that any of A, B, and C, or any combination thereof, can be omitted and disclaimed singularly or in any combination. If a component D is separately described, it may be combined with any foregoing combination.

Recitation of ranges of values herein are merely intended to serve as a shorthand method of referring individually to each separate value falling within the range, unless otherwise indicated herein, and each separate value is incorporated into the specification as if it were individually recited herein. For example, if a concentration range is stated as 1% to 50%, it is intended that values such as 2% to 40%, 10% to 30%, or 1% to 3%, etc., are expressly enumerated in this specification. These are only examples of what is specifically intended, and all possible combinations of numerical values between and including the lowest value and the highest value enumerated are to be considered to be expressly stated in this disclosure.

As used herein, “monitoring health and wellness” comprises any means from which the health and wellness of a subject can be monitored. Examples include, but are not limited to, the use of digital recordings from any device which records data related to health or wellness, coupled with metabolic profiles as exemplified below in the Examples. In some embodiments, the monitoring of health and wellness further provide for outputs on how a subject can adjust their lifestyle to improve their health and wellness. A method or action to improve a subject's health and/or wellness may be considered an “intervention”. Such methods, actions or interventions may be considered a “perturbation” when it is expected to affect data being collected across a population or from an individual subject, or may be referred to as a way to adjust life style to improve health and/or wellness.

As used herein, ways to adjust life style to improve health and/or wellness, such as those determined by sensors digital recording coupled with metabolic profiles as provided herein, include, but are not limited to, ways to change diet and/or exercise to maintain health, ways to revert a trend towards development of disease, ways to revert a trend to mitigate a disease, use of supplements, probiotics, nutraceuticals, and/or medications, treatments or therapy, prophylactic treatments or therapy, and other known ways. Supplements or nutraceuticals may be customized for any particular individual or group.

As used herein, “treatment,” “therapy” and/or “therapy regimen” refer to the clinical intervention made in response to a disease, disorder or physiological condition manifested by a subject or patient or to which a subject or patient may be susceptible. The aim of treatment includes the alleviation or prevention of symptoms, slowing or stopping the progression or worsening of a disease, disorder, or condition and/or the remission of the disease, disorder or condition.

As used herein, the terms “prevent,” “preventing,” “prevention,” “prophylactic treatment” and the like refer to reducing the probability of developing a disease, disorder or condition in a subject, who does not have, but is at risk of or susceptible to developing a disease, disorder or condition.

The term “effective amount” or “therapeutically effective amount” refers to an amount sufficient to effect beneficial or desirable biological and/or clinical results.

As used herein, the term “administering” an agent, such as a therapeutic entity to an animal or cell, is intended to refer to dispensing, delivering or applying the substance to the intended target. In terms of the therapeutic agent, the term “administering” is intended to refer to contacting or dispensing, delivering or applying the therapeutic agent to a subject by any suitable route for delivery of the therapeutic agent to the desired location in the subject, including delivery by either the parenteral or oral route, intramuscular injection, subcutaneous/intradermal injection, intravenous injection, intrathecal administration, buccal administration, transdermal delivery, topical administration, and administration by the intranasal or respiratory tract route.

As used herein, the term “biomarker” refers to any objective measure that captures what is happening in a cell or an organism (e.g., a subject) at a given time. In some embodiments, the biomarker comprises a naturally occurring biological molecule present in a subject at varying concentrations useful in predicting the risk or incidence of a disease, disorder or a condition. For example, the biomarker can be a protein present in higher or lower amounts in a subject at risk for a disease or condition. The biomarker can include nucleic acids, ribonucleic acids, polypeptide, sugars, polysaccharides, lipids, carbohydrates, and the like used as an indicator or marker for the disease or condition. For example, in some embodiments, the biomarker may comprise glucose (e.g., glucose levels as measured by continuous monitor). In other embodiments, the biomarker comprises a lipoprotein such as HDL, LDL, etc. for the monitoring of, for example, cardiovascular disease risk, and the like. A biomarker may also comprise any naturally or non-naturally occurring polymorphism (e.g., single-nucleotide polymorphism [SNP]) present in a subject that is useful in predicting the risk or incidence of an undesired disease or condition. Biomarkers may also be non-molecular in nature, and can include everything from physiological measures such blood pressure, heart rate, EEG, EKG, arrhythmias, sleep disorders to metabolic studies, information in x-ray/NMR/MRI findings, to histologic and genetic tests of blood and other tissues. As used herein, biomarkers may further include additional genetic data, such as those available through companies like 23&Me™, Ancestry™, gut microbiome data, etc. Such data is further measured and incorporated into the metabolic profile and, optionally, in the metabolomic data, wherein any metabolic defects are also mapped to a possible genetic variation or gut bacterial enzymatic reactions adding net influences of genome and gut microbiome on metabolome. Biomarkers are measurable, but do not necessarily define how a subject feels or functions. One or more biomarkers collected from an individual can be compiled to create a metabolic profile. Biomarker data collected from a plurality of individuals can be compiled to create metabolomic data that can be stratified and used in methods described herein, such as to help diagnose a specific individual or specific group of individuals based on their metabolic profile.

The term “biological sample” as used herein includes, but is not limited to, a sample containing tissues, cells, and/or biological fluids isolated from a subject. Examples of biological samples include, but are not limited to, urine, saliva, respiration (e.g., for use in a breathalyzer), other body fluids such as plasma, tears, bile, spinal fluid, mucus, lymph, serum, blood, and the like, skin or tissue, biopsies, fecal samples among others. A biological sample may be obtained directly from a subject (e.g., by blood or tissue sampling) or from a third party (e.g., received from an intermediary, such as a healthcare provider or lab technician).

As used herein, the term “metabolomics data” and “metabolome data” and “metabolic data” refers to the compiling and grouping of one or more of (i) data collected from one or more subjects as determined by a biological sample as provided herein, and/or (ii) additional genetic data and/or gut microbiome data, and the like, that are compiled such that any metabolic defects are also mapped to a possible genetic variation or gut bacterial enzymatic reactions adding net influences of genome and gut microbiome on metabolome.

As used herein, the term “device” or “digital health wearable” or “sensor” and such are used interchangeably and include those devices that can be worn, implanted, used and/or carried by a subject and are capable of detecting and/or recording and/or processing, and/or transmitting data (e.g., digital health data). In some embodiments, the device is capable of compiling, analyzing, and providing an output within the device itself. In other embodiments, the device is capable of doing one or more of compiling, analyzing, and providing an output. In yet other embodiments, the device is capable of communicating, either through wired or wireless transmission, recorded data to one or more other devices, the cloud (Internet), networks, and the like. Devices may be wearable, non-wearable or implantable.

In some embodiments, the devices may be wearable. Examples of wearable devices include, but are not limited to, (i) commerically available wearable biosensors such as Phillips™ brand devices (ii) smart watches (e.g., Apple™ watch, Google™ watch, Fossil™ Carlie hybrid watch); (iii) smart jewelry (e.g., Oura™ smart ring, Bellabeat™ Leaf Urban (necklace or bracelet), Withings Steel HR™, Bellabeat™ Leaf Chakra necklace, and the like); (iv) wearable heart rate, ECG, and/or temperature monitors; (v) fitness trackers (e.g., Fitbit™, Garmin™, etc.); (vi) smart clothing, such as smart shoes, smart sleepwear, smart activewear, smart casual wear, smart socks, etc. (e.g., Wearable X™, Levi's™ Google Jacquard, Ambiotex™, Komodo™, Under Armour™, Owlet™, Neviano™, Hexoskin™, Sensoria™, Athos™, Siren™, Ministry of Supply™, Neopenda™, Seismic™, etc.); (vii) eyewear (e.g., Google™ glasses, Spectacles™, etc.); (viii) headwear (e.g., iBand™ smart wireless Bluetooth headband, Muse™ smart headband, Melon™ smart headband, Thync Relax™ smart headband, Luma™ Active smart headband, Aurora by iWinks™, Spree™ bluetooth Fitness monitor; and the like.

In other embodiments, the device may be non-wearable (i.e., able to be carried and/or used by a subject to measure biomarkers). Examples of non-wearable devices include, but are not limited to smartphones, tablet computers, magic pens (e.g., such as those that can measure coition), etc., use of software (apps) on devices such as smartphones and tablet computers that can measure for a biomarker as provided herein, and the like.

In other embodiments, the device may be implantable. Examples of implantable devices include, but are not limited to, (i) Glucose monitors (e.g., Dexcom/Freestyle Libre); (ii) Insertable cardiac monitors for measuring biomarkers such as EKG, ECG, etc.; (iii) implantable monitors for monitoring brain function (e.g., EEG) (e.g., for Parkinsons via tremors in limbs); (iv) electronic tattoos (e.g., http://www.medicalautomation.org/2011/10/electronic-tattoos-for-biomonitoring/).

The term “disease” as used herein includes, but is not limited to, any abnormal condition and/or disorder of a structure or a function that affects a part of an organism. It may be caused by an external factor, such as an infectious disease, or by internal dysfunctions, such as cancer, cancer metastasis, metabolic disorders, and the like.

As used herein, the terms “subject,” “individual,” and “patient” are used interchangeably herein and refer to both human and nonhuman animals. The term “nonhuman animals” of the disclosure includes all vertebrates, e.g., mammals and non-mammals, such as nonhuman primates, sheep, dog, cat, horse, cow, chickens, amphibians, reptiles, and the like. The methods and compositions disclosed herein can be used on a sample either in vitro (for example, on isolated cells or tissues) or in vivo in a subject (i.e. living organism, such as a patient).

Unless otherwise defined, all technical terms used herein have the same meaning as commonly understood by one of ordinary skill in the art to which this disclosure belongs.

The present disclosure is based, in part, on the discovery by the inventors of devices, systems, and methods for connecting metabolomics measurements to big data being generated by sensors captured in databases to enable monitoring of metabolic health at molecular level to map disruptions in biochemical processes in each individual that can inform about health, basis of disease, subtype of disease and ways to correct for such defects tailored for each individual. The use of sensors combined with molecular signatures is a far more powerful approach for providing precision health and precision medicine approaches tailoring best treatment for each individuals or for adopting personalized approaches to sustain health and prevent, delay, or treat disease.

In certain aspects, the present disclosure relates to connecting metabolomics measurements to big data being generated by digital monitors and sensors captured in databases to enable monitoring of metabolic health at molecular level to map disruptions in biochemical processes in each individual that can inform about changes in health, molecular basis of disease, subtypes of disease and ways to correct for such defects tailored for each individual. The use of sensors combined with molecular signatures can be a far more powerful approach for providing precision health and precision medicine approaches tailoring best treatment for each individual.

### Metabolomics Data Couples to Digital Monitors Enables Precision Health

### A. Concept:

Recent developments in high-throughput omics screening technologies and their broad application in biomedical research, the use of medical records and the development of digital monitors and sensors that enable continuous collection of data on health is all creating highly valuable information about health of individuals and in real time. This combined with use of machine-learning methods and AI technologies provides great promise for optimizing health for each individual. New fields are emerging based on use of such big data including precision health, precision nutrition and precision medicine.

The development of continuous glucose sensors by several companies such as Abbotts Laboratory Free Style Libre, Dexcom and others enables measurement of glucose around the hours of the day and night enabling an individual to learn about their eating and life style habits that can influence their glucose levels to better manage diabetes and other glucose related diseases. The use of sensors for sleep patterns are many from watches to bands and rings placed on head or wrist enable tracking of sleep patterns and disruptions of sleep that could be linked to disease including for example neuropsychiatric diseases. Blood pressure (BP) monitors come in different types including continuous monitoring throughout a day. Heart monitors ECG and EKG are other examples of monitors for cardiovascular health. An electrocardiogram records the electrical signals in heart. It's a common and painless test used to quickly detect heart problems and monitor one's heart's health.

Electrocardiograms—also called ECGs or EKGs—are often done in a doctor's office, a clinic or a hospital room. ECG machines are standard equipment in operating rooms and ambulances. Some personal devices, such as smart watches, offer ECG monitoring. Electroencephalographic EEG brain monitoring headsets detect electrical brain waves through electrodes placed in an array along the patient's scalp. Medical and neuroscience researchers have established what normal brain activity looks like. Patients with epileptic seizures, sleep disorders and other disturbances display abnormal activity on the monitor of an EEG. EEG monitoring is used to detect the activity required to make or confirm a diagnosis. Headbands, magic pens, digital voice are among few of signals being captured to inform about brain and cognitive function. Accordingly, the devices, systems and methods of the present disclosure provide use of a device as defined herein to detect and/or record and/or transmit and/or output instructions based on biomarker data provided by a subject.

Applications of metabolomics tools and patient's metabolic profiles have been developed as a readout to inform about net influences of variations in the genome, environmental exposures, effect of dietary habits, lifestyle and gut microbiome influences on overall metabolic states in health and in disease. Measuring thousands of chemicals is now possible to expand knowledge about metabolic health beyond glucose, cholesterol and a small panel of chemicals measured during annual checkups. Such data can capture current physiological state of an individual that can further inform about health, deviations in acute and chronic diseases and treatment outcomes. Multiple applications can emerge from using this data including development of additional sensors to monitor health, environmental toxins, disease sub classification and reclassification, pathways and mechanisms implicated in disease and selection of most appropriate diet and medications for individuals.

One embodiment of the present disclosure relates to connecting metabolomics measurements to big data being generated by devices as described herein (including any digital monitors and sensors) captured in databases to enable monitoring of metabolic health at molecular level to map disruptions in biochemical processes in each individual that can inform about basis of disease and ways to correct for such defects tailored for each individual.

Metabolomics is the comprehensive study of the metabolome, the repertoire of biochemicals (or small molecules) present in cells, tissues, body fluids and organisms. The study of metabolism at the global or “-omics” level is a rapidly growing field that has the potential to have a profound impact upon medical practice. At the center of metabolomics, is the concept that a person's metabolic state provides a close representation of that individual's overall health status. This metabolic state reflects what has been encoded by the genome, and modified by diet, environmental factors, and the gut microbiome. The metabolic profile provides a quantifiable readout of biochemical state from normal physiology to diverse pathophysiologies in a manner that is often not obvious from gene expression analyses. Today, clinicians capture only a very small part of the information contained in the metabolome, as they routinely measure only a narrow set of blood chemistry analytes to assess health and disease states. Examples include measuring glucose to monitor diabetes, measuring cholesterol and high density lipoprotein/low density lipoprotein ratio to assess cardiovascular health, BUN and creatinine for renal disorders, and measuring a panel of metabolites to diagnose potential inborn errors of metabolism in neonates.

The inventors show that the narrow range of chemical analyses in current use by the medical community today may be replaced by analyses that reveal a far more comprehensive metabolic signature. This signature is expected to describe global biochemical aberrations that reflect patterns of variance in states of wellness, more accurately describe specific diseases and their progression, and greatly aid in differential diagnosis. Such metabolic signatures: (1) provide predictive, prognostic, diagnostic, and surrogate markers of diverse disease states; (2) inform on underlying molecular mechanisms of diseases; (3) allow for sub-classification of diseases, and stratification of patients based on metabolic pathways impacted; (4) reveal biomarkers for drug response phenotypes, providing an effective means to predict variation in a subject's response to treatment (pharmacometabolomics); (5) define a metabotype for each specific genotype, offering a functional read-out for genetic variants; (6) provide a means to monitor response and recurrence of diseases, such as cancers; (7) describe the molecular landscape in human performance applications and extreme environments. Importantly, sophisticated metabolomic analytical platforms and informatics tools have recently been developed that make it possible to measure thousands of metabolites in blood, other body fluids, and tissues.

Metabotyping is currently used to screen for inborn errors of metabolism. Metabolism data and inclusion of a targeted metabolic profile in blood has led to the identification of many inborn errors of metabolism (IEMs), a group of monogenetic metabolic disorders that can be lethal in newborns, or result in irreversible organ damage, if not diagnosed and treated swiftly. However, if IEMs are recognized by early screening, many can be controlled with life-saving nutritional supplements and dietary interventions. Using current metabolite profiling platforms, which can now survey thousands of metabolites in microliter quantities of neonatal blood, we anticipate an enormously expanded scope of IEM diagnosis and the discovery of previously unrecognized genetic diseases in the near-term.

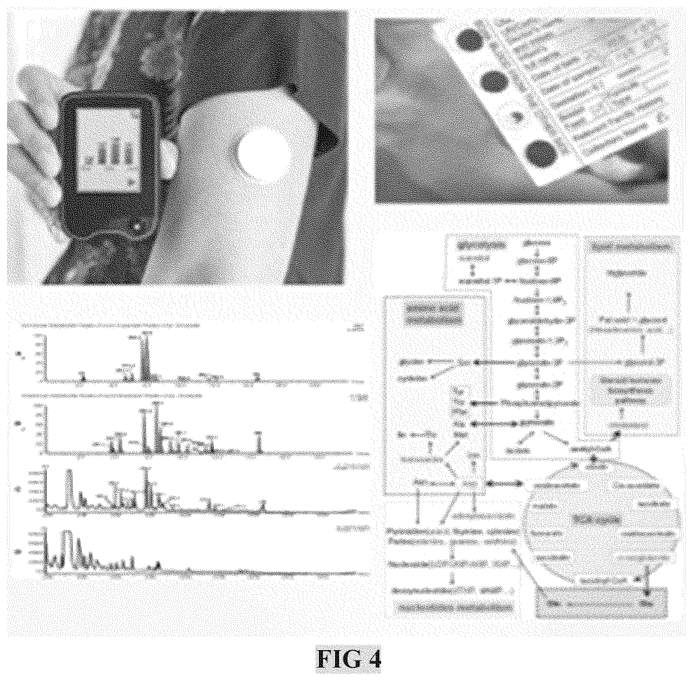

It is believed that “inborn errors of metabolism” are “merely extreme examples of variations of chemical behavior which are probably everywhere present in minor degrees” and that “chemical individuality predisposition to and immunities from the various mishaps which are spoken of as diseases”. Population based studies collected demographic, health and life-style related information from thousands of individuals from the general population, and bio-banked samples of blood, urine, and other body fluids are now analyzed using genomics, transcriptomics, proteomics, metabolomics and other large scale omics technologies. It has been shown that genetic predisposition interacts through intermediate metabolic phenotypes with environmental factors and lifestyle choices in the pathogenesis of complex disorders. FIG. 4 gives an example of a genetically influenced metabotype (GIM). To date, over 150 such GIMs have been discovered and are now being used to dissect the genetic and environmental factors that trigger complex disorders. A comprehensive blueprint of human metabolic pathways and linkage to genes and their expression, such as the global human metabolic network that had been created by the systems biology community, would inform strategies for modifying dysregulated metabolites in a rational and targeted manner, potentially using drugs that are pre-existing and safe—as suggested by recent genome-wide association study (GWAS) findings.

Advancements in the study of metabolites and the development of the field of metabolomics has resulted in part from innovative advances in scientific instrumentation and advances in computational resources available. The continued development of chromatography coupled to mass spectrometry (MS) and nuclear magnetic resonance (NMR) spectroscopy have advanced our capabilities from monitoring only a small number of metabolites in a traditional hypothesis-testing study, to being able to simultaneously quantify hundreds to thousands of metabolites in a biological sample with an analysis time of less than 20 min (Cajka and Fiehn 2014; Fiehn 2016). This enormous new analytical capability has led to the generation of novel and completely unanticipated hypotheses (Dunn et al. 2011)—effectively shining light in places where nobody previously thought to look. This non-targeted approach is referred to as metabolic phenotyping (“metabotyping”).

Importantly, metabotyping can provide important data not only on the metabolites present in a complex biological mixture, but also reveal molecular interactions that contribute to metabo-regulatory processes in cells and tissues. Only through the application of such holistic approaches, as a first step, can the complete biological interactome be defined in relation to human phenotypes. Once specific metabolic markers are identified, further studies applying targeted assays can be performed in attempt to validate findings and test novel hypotheses that emerge. Recent translational studies illustrate how metabotyping can lead to new and fundamental biomedical discoveries.

The current capabilities for holistic metabolic profiling apply liquid chromatography and gas chromatography coupled to mass spectrometry (GC-MS and LC-MS), as well as NMR spectroscopy. The stability and reproducibility of these platforms is hugely important in large-scale cohort studies and recent research has shown the capability now exists for robust and high-quality data generation. This has allowed us to move from small-scale studies to large-scale investigations that may include thousands of samples. Indeed, a number of ‘Phenome Centers’ are being developed to provide the infrastructure and resources required to support large-scale studies (e.g., the National Phenome Centre in London; the Phenome Centre Birmingham UK; six NIH Regional Comprehensive Metabolomics Resource Cores in the USA and large consortia, such as Alzheimer Disease Metabolomics Consortium). Standardization of analyses performed at these centers will be a challenge, but recognized as essential to allow data to be comparable and integratable, as no single center currently provides the capacity for studies of the scale envisioned for the PMI Cohort. Without standardization there will be no ability to take data across sites and compare/integrate. These new forays have allowed knowledge from metabolic profiling studies to be synergistically applied to studies where only genomic and transcriptomic data were previously available.

Although this combined ‘omics strategy is being applied to enhance metabolic phenotyping in a holistic approach with relative quantification data created, the application of multiple (semi)-targeted assays for each sample provides a robust approach for absolute quantification of more limited metabolite panels (hundreds, not thousands). For example, the company Biocrates commercially supplies kits for analysis of targeted areas of metabolism. The Biocrates p180/Q500 kits measures amino acids, biogenic amines bile acids and lipids, and gut microbiome metabolites and has been shown to be useful for evaluating dry blood spots. Nightingale Health's NMR based metabolomics platform measures close to 200 metabolites mainly lipoproteins. The company has profiled over 1 million individuals to date including UK Biobank and has developed phone Aps that capture risk for developing cardiovascular disease and diabetes. Metabolon uses complementary LC and GC methods to measure close to 1000 metabolites and hundreds of studies has shown the power of the platform to inform about disease.

Each metabolomics platform provides advantages in interlaboratory use and limitations in terms of the number of metabolites studied (where holistic untargeted approaches are far more comprehensive) and the type of quantitative information obtained (untargeted MS-based profiling provides relative differences in metabolite levels, targeted profiling provides absolute levels). Importantly, there is no single assay that can detect all metabolites present in a given sample, nor do we even currently know how many distinct molecules can be quantified. At present, the metabolome can be viewed as a biomedical frontier with important opportunities to inform on systemic processes that provide the underpinning for the coming revolution in precision medicine. Employing a combination of hydrophobic and hydrophilic chromatography with both positive—and negative-ion monitoring mass spectrometric detection, it is possible to detect 3000-4000 distinct molecules using as little as 1-20 μl of plasma. To take advantage of such recent analytical breakthroughs, the metabolomics community strongly recommends a combination of targeted and untargeted strategies to maximize information that can now be obtained from of an individual patient sample or sample cohort.

Metabolic profiling will improve as analytical approaches, compound identification, ion mobility, and derivatization agents are further developed. Major drives in metabolomics toward greater metabolome coverage, higher sensitivity and even smaller sample volumes are envisioned to culminate in capabilities for single cell metabolomics. Bottlenecks are now evident in reconciling data obtained from different laboratories, though significant efforts to eliminate these are underway globally, including enhancements in data standardization, quality assurance and high-confidence metabolite identification. Mineralizing metabolomics will lead to development of additional sensors to that of glucose and where tens to hundreds of chemicals can inform about some aspects of health.

The type of sample being studied is always a key component in any experimental design and the choice of sample is obviously dependent on the biological question. In large-scale cohort studies, biofluids (e.g., serum, plasma, urine, saliva, breath, stool) are most commonly employed because of their relative ease of collection, preparation and storage. No single biofluid is appropriate for all studies and the biological question defines the biofluid—oral cavity diseases match to using saliva, gut microbiome and kidney diseases match to urine whereas cardiovascular diseases and drug PK studies match to serum or plasma. K-EDTA-plasma is recommended for blood collection. Importantly the metabolome analyzed has to be representative of the metabolome collected; as metabolism operates on timescales of seconds and minutes in enzyme-containing samples and as metabolites can be chemically degraded at high temperatures or extreme pH values, it is essential that samples are collected and processed quickly to minimize any changes in metabolite composition, both qualitatively and quantitatively. Traditionally, blood or urine samples have been collected in clinic and processed by trained staff for storage. However, this is time-consuming and costly for patients and researchers in cohort studies where travel costs, staff costs, and laboratory costs are required. One alternative is for participants in large precision medicine initiatives is to collect samples in their homes and send them frozen via express mail to a central biobank facility, thus eliminating the need for participants to travel and minimizing the efforts/costs of a trained staff. The collection of dried blood or dried urine spots on absorptive materials, followed by ambient temperature delivery by post to storage facilities, offers the potential for low-cost and larger-scale global metabotyping efforts [see cityassays.org.uk 2014 for an example for targeted analysis of vitamin D]. This emerging approach is currently being explored and may be applicable for studies during the next few years. Notably, analysis of dried blood spots is already the standard for newborn screening of inborn errors in metabolism and applied in many drug metabolism studies. The analysis of dried blood and urine spots has had limited application to holistic metabolic profiling studies as yet, but validation and adoption of this approach is anticipated.

In one embodiment, metabolic measurements or signatures are derived by providing a drop of blood on filter paper (e.g., as is being done for newborn screening to detect metabolic defects called inborn errors of metabolism), through a capillary device where a drop of blood is absorbed by, for example, a cotton swab, as in mitra device or similar approaches, Tasso™ devices (https://www.tassoinc.com/) or by providing blood samples in clinical setting as is routinely done. The filter paper enables continuous monitoring of chemicals by sending these filter papers to specialized metabolomics labs that can extract chemicals from blood and analyze for tens to thousands of chemicals using, for example, mass spec and or NMR based approaches. In other embodiments, the methods provide using a biological samples, such as urine or saliva, breath skin for analysis instead of blood samples.

In other embodiments, the methods further comprises adding genetic data, for example through companies like 23&Me™, Ancestry.com™, and the like to allow for further mapping of metabolic defect(s) to a possible genetic variation. In yet other embodiments, the methods comprise obtaining a biological sample, such as a fecal sample, for metabolomics as well as for metagenomics analysis to allow for the assessment of gut microbiome changes contributing to human health and disease. The metabolome is influenced by both genome and gut microbiome hence provides a readout of these influences on human health.

### MetaboSensor Database

In other embodiments, the present disclosure provides for a network in which multiple users import their collected metabolic data to a main database, which may be stored in a central location or in a distributed manner, which may be referred to herein as the MetaboSensorDB. The data from the multiple users can then be compiled, analyzed, stratified and then compared to an individual person such that the user receives an output comparing their metabolic data to that of an identifiable group of other users (or subset of those users). First, such data can inform about health preservation of health and prevention of disease in a personalized way by life style interventions. Additionally, such collected data can also be used to screen for novel drugs, therapies, biomarkers, etc. for the development of novel drugs and therapies for the prevention, identification, and treatment of identified metabolic disorders in subgroups of patients who share similar characteristics captured by digital and biochemical recording.

In one aspect, disclosed is a method of monitoring the health and wellness of an smaller group of individuals, and even a single individual, which involves creating a database of digital health data and metabolomic data over a period of time. The database may also include other omics data such as microbiomic data and/or genomic data from the individual. This database may be used to monitor changes to the individual's health or wellness, identify the impact of interventions or therapies, and inform decisions regarding possible interventions or therapies for the individual.”

Thus, in another aspect, disclosed is a method of improving the health and/or wellness of a subject. A first step is to create a main database (referred to herein as the MetabosensorDB) by collecting continuous digital health data and metabolomic data for a population of individuals over an extended time period where at least one perturbation, and preferably multiple perturbations (diet, lifestyle, etc.) are applied. The population of individuals is preferably diversely representative.

Within the MetabosensorDB, standard multivariate statistical methods are used to group members of the population into identifiable health stratification groups such as metabotypes (metabolic health phenotypes or MHPs) and digital health phenotypes (DHPs) in MetaboSensorDB. The method then comprises the steps of:
(d) Collecting digital health and metabolic data for a subject over a shorter time period, for example, a single 24 hour period (or shorter or longer periods);
(e) Comparing the subject's digital health phenotype (DHP) and metabolic health phenotype (MHP) to see which health stratification group in the MetaboSensorDB the subject matches with;
(f) Apply a perturbation to the subject, which perturbation is associated with a desired health or wellness outcome for individuals in the health stratification group matched by the subject.

In some embodiments, the subject may continue to collect digital health data and/or metabolomic data and determine the effect of the perturbation on the DHP and/or MHP of the subject, which is added to the MetabosensorDB.

In some embodiments, if the subject experiences a change in DHP and/or MHP which is different than the matched health stratification group, the subject may be moved to another health stratification group, or a subgroup may be identified. A statistical multivariate analysis, using a method the same or different than was originally used to create the health stratification groups, may be reapplied to the database with the new data included. The fact that the subject had a different experience or result than other individuals in the group should result in the subject matching more closely with another group, or may result in the creation of a new group or subgroup. The subgroup may consist of the subject alone.

In some embodiments, the MetaboSensorDB also comprises one or both of genomic profile data and microbiome profile data from the population of individuals. The subject may then also collect and compare such genomic profile data and/or microbiome profile data in the matching step to find a matching health stratification group.

### Digital Health Status Groupings

A digital health status grouping (DHSG) method described herein, comprises converting digital device output over an extended period of time into distinct digitally-derived physiological categories or DHSGs.

An external perturbation (changed diet, glucose tolerance test, physical stress, exercise regimen, mental stress, drug or supplement stress, disease episode, etc.) be applied over an extended period of time and that biological samples (blood, urine, etc.) be collected over that time for comprehensive metabolomics (or other omics) analysis. To make this concept generalizable, these longitudinal stress/digital health/metabolomics tests must be conducted on a number of individuals (different ages, sex, ethnicity, weight, health conditions, etc.). By collecting both digital health and metabolomics data on a sufficient number (>100 or less) of people using the same digital health monitoring and the same metabolomic monitoring approaches described here, it should be possible to identify individuals who share similar digital health phenotypes (DHPs), or individuals who share similar metabolite health phenotypes (MHPs).

It would also be possible to identify individuals who respond to similar perturbations in the same way. This database of DHPs, MHPs and DHP/MHP responses would be of considerable value for implementing true precision health or precision nutrition. In particular, after its construction, it would allow other subjects to simply take a single metabolomics test or take a single battery of digital health tests and to use these data (and the database) to determine which DHP or MHP they belong to and what their responses to different diets or drugs or other stressors would be. Such a classification would also allow personalized dietary, pharmaceutical, or lifestyle advice (e.g., Mediterranean diet combined with an exercise program emphasizing weight training) to be provided to the subject to correct or improve their DHP and/or MHP—if it is found to be unhealthy or the individual otherwise desires enhancement or improvement. Taking only one test avoids the time and expense of a testing program involving dozens of blood and urine tests over several weeks. However, single time-point tests may not always be accurate. If a single user chooses to collect digital measurements over a certain period of time, they may further refine or determine exactly which DHP or MHP they match (e.g., exercise response) to make more precise refinements to their lifestyle or diet. Continual monitoring via digital measurements and/or additional metabolite testing may allow more precise stratification, and more precise engagement with other users to learn, discover, and share what lifestyle/diet interventions may be personally beneficial, neutral, or harmful to them.

The concept of DHSGs can be extended from the longitudinal monitoring of one or a plurality of individuals under different stressors to the cross-sectional monitoring of a larger group of individuals who differ based on their inborn physiology, disease state, or their habitual lifestyle or dietary habits. For example, a sample of 100 individuals may yield 30 to 40 individuals that each fall into 3 or 4 distinct DHSGs (sleep pattern, mood, mental performance, blood pressure, heart rate, etc.). In other cases, the sample may include less than 100 individuals.

The same kind of metabolomic analyses for these DHGs may be done to identify metabolite associations, exposome associations, or metabolite “drivers” that appear to cause people, for instance, to have high blood pressure or lower heart rates or better mental performance. Cross-sectional digital health monitoring over a larger number of people may also allow the identification of more distinct categories or larger numbers of categories (going from 3 to 5 categories, such as very low, low, medium, high, very high). These digital health phenotypes (DHPs), which can be easily derived from comparing and classifying digital health devices for large numbers of people, could be used to classify individuals into specific DHPs. Likewise, the corresponding metabolomic profiles or metabolic health phenotypes (MHPs) collected from population studies could also be used to classify individuals into specific MHPs. Using data collected about an individual's diet, lifestyle, age, sex, ethnicity, weight, etc., it should also be possible to correlate specific DHPs with MHPs along with certain habitual diets, drugs, or lifestyles. This would also allow the creation of a database that would be of considerable value for precision health or precision nutrition. In particular, it would allow other subjects to simply take a single metabolomics test or take a single battery of digital health tests to determine which DHP or MHP they belong to and what their responses to diet or drugs would be. Such a classification would also allow personalized dietary, pharmaceutical, or lifestyle advice (e.g., Mediterranean diet combined with an exercise program emphasizing weight training) to be provided to the subject to correct or improve their DHP and/or MHP—if it is found to be unhealthy or the individual otherwise desires enhancement or improvement. Taking only one test avoids the time and expense of a testing program involving dozens of blood and urine tests over several weeks. However, single time-point tests may not always be accurate. If a single user chooses to collect digital measurements over a period of time, they may further refine or determine exactly which DHP or MHP they match (e.g., exercise response) to make more precise refinements to their lifestyle. Continual monitoring via digital measurements and/or additional metabolite testing may allow more precise stratification, and more precise engagement with other users to learn, discover, and share what lifestyle interventions may be personally beneficial, neutral, or harmful to them.

### EXAMPLES

The following Examples are provided by way of illustration and not by way of limitation.

### 1. Accessible Data

Earlier work established foundations for a new field “Pharmacometabolomics”, which parallels and informs “Pharmacogenomics” and in which metabolic profiles of individuals are used to inform about treatment outcomes. The inventors have mapped metabolic effects of commonly used medications including simvastatin, anti-platelet therapies (aspirin, clopidogrel), anti-diabetic (metformin) anti-hypertensive drugs (atenolol and thiazide), antidepressants (3 SSRIs sertraline escitalopram citalopram, SNRI duloxetine and ketamine), mood stabilizers (lithium), atypical antipsychotics (olanzapine, risperidone and aripiprazole), ADHD treatment (atomoxetine) among other medications. The inventors defined signatures that correlate with therapeutic benefit, signatures that inform about disease heterogeneity and treatment outcomes. This vast data is available as base to explore how metabolomics data can inform about response to treatment.

Further work is being performed defining in large studies metabolic alterations that inform about depression anxiety heterogeneity and mechanisms, sleep disruption, fatigue, effects of currently used therapies and variation in response to treatment. Metabolomics data is connected to genetics and imaging data to learn more deeply about each patient. Adding digital recording from patients such as watch for recording sleep patterns, a glucose monitor, EEG, a CVD or diabetes rick factor determined by Nightingale profiles can all lead to far more detailed mapping about each individual health and disease and optimize treatment.

Using eight metabolomics and lipidomics platforms, the inventors have mapped a biochemical trajectory of Alzheimer's disease defining disease subtypes, connecting peripheral and central metabolic changes, connecting genome and metabolome to define genetic base for biochemical changes, and building a first molecular atlas for disease using big multi omics data. Over 40,000 metabolic profiles generated longitudinally and from thousands of patients and from community studies to learn more deeply about each patient have been created.

An initiative related to the gut microbiome and Alzheimer's disease (AD) includes leadership from the gut microbiome, AD, depression and metabolomics fields with a mission to define a possible role for gut microbiome in AD and other neuropsychiatric diseases and for defining gut brain chemical axis of communication. This project will set the stage for identifying measuring and tracking gut bacterial products that impact human health. Adding digital recording from patients such as watch for recording sleep patterns, a glucose monitor, EEG, a CVD or diabetes rick factor determined by Nightingale profiles can all lead to far more detailed mapping about each individual health and disease and optimize treatment.

The inventors have access to thousands of samples from Rotterdam study. The first 1000 subjects have been profiled where the metabolomics data is being connected to genetic, diet, gut microbiome, imaging and clinical data and learning about cognitive function. Recent approval was granted to profile 8000 samples from Framingham Heart Study generation 2 exams 7,8,9 and Omni Generation 1 Cohort, and Omni Generation 2 Cohort. Further work provides for the profiling of over 500 brains and matching blood samples to learn about metabolic aberrancies that compromise brain health. Metabolic profiles of 120,000 participants in UK Biobank where signatures related to CNS diseases and before data goes public are being investigated. The inventors are also performing lipidomic profiling of over 10,000 subjects (AIBL ADNI and Australia community studies) learning about lipid identities, genetic influences and predisposition to metabolic and other diseases.

We are using biochemical data, risk indices calculated, genetic gut microbiome data and coupling all of this to digital data as described above for a deeper understanding of each individual and what works best for each.

Previous work developed atlases that connect genome and metabolome and inform about genetic influences on human metabolism. These atlases are now being expanded to construct first atlases for human diseases where public genetic data, large metabolomics data and clinical data are used to inform about disease mechanisms, novel targets for drug design and for repurposing of drugs that currently exist. Construction of an atlas for depression is underway and has already provided new insights.

### 2. Sleep Disruption Mood Changes and Metabolism

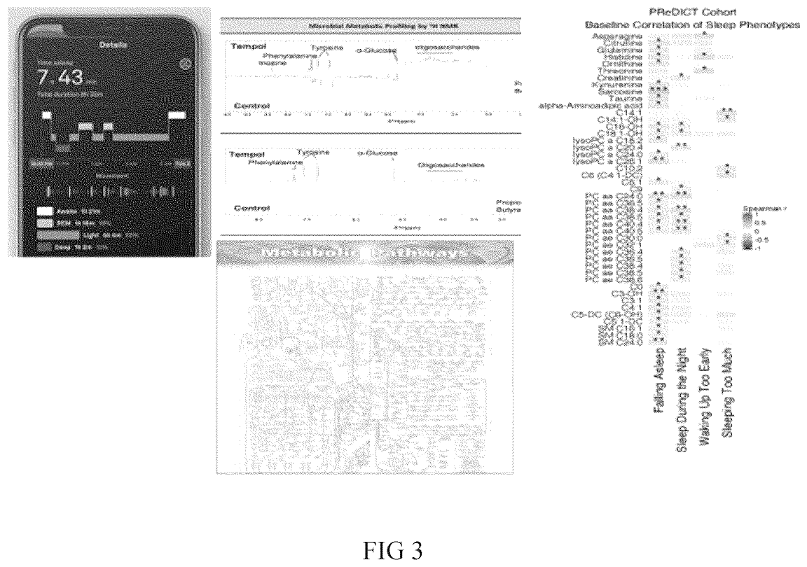

One aspect of the present disclosure allows for the monitoring of sleep disruption mood changes and its linkage with metabolism, and in some cases, providing an output that provides interventions as needed to aid with the subject's health and wellness. In one embodiment, and as shown in FIG. 3, this is accomplished by connecting digital recording from a device capable of capturing and recording data (e.g., a watch, smart device (e.g., smart phone), ring, monitor, etc.) that monitors sleep and disruptions in sleep patterns of a subject. The subject then also provides a biological sample from which a metabolic profile is determined. In some embodiments, clinical data on mood and anxiety (e.g., a questioner through phone AP or from clinical evaluation, etc.) is added to allow for a better understanding in groups of subjects with sleep disruption patterns molecular basis for this disruption. This biochemical information thereby leads to actionable intervention such as change in life style that reverts metabolic defects and/or supplements and/or drugs that help correct metabolic defect.

One example of method for incorporating the coupling of digital recordings from a device as provided herein with metabolic profiles to inform about molecular basis for sleep disruption comprises, consists of, or consists essentially of: (a) obtaining digital recording(s) from the device to inform about sleep patterns; (b) generating a metabolic profile from a biological sample (e.g., a blood sample) of a participant such as by using NMR or MS based metabolomics platform; (c) analyzing the metabolic profile informed by clinical data from subjects who have problems in sleep patterns such as difficulty falling asleep, waking up in the middle of the night, waking up early and not being able to go back to sleep or in people who sleep too much; and (d) providing an output that reveals the underlying metabolic defects and instructions for correcting by modifying life style, diet, of supplements or medications and the like. The problem in sleep patterns may be related to conditions such as depression and/or anxiety.

### 3. Blood Glucose Measurements

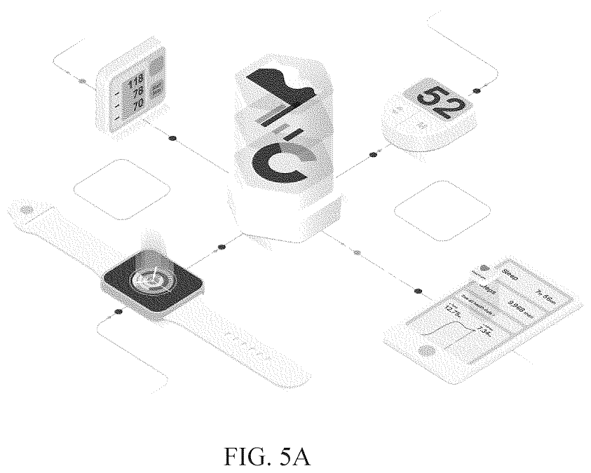

As an example, a glucose continuous monitor can indicate fluctuations in levels of blood glucose throughout the day. Peaks of levels of glucose after meals can indicate a personal response to diet and which diets might be best for an individual. Also high levels of glucose early in the morning vs later in day can indicate metabolic aberrances including in liver function. Adding a metabolomics profile through collected biologicals samples (e.g., blood, urine, saliva, or fecal sample) can provide biochemical insights about pathways affected in each individual (see, e.g., FIGS. 4 and 5A/5B). That reflect the signal from a sensor that is a biochemical basis for disease in that individual or the response to a medication. This profile can be further linked to genetic information or gut microbiome data to tell if problem noted in metabolism has a genetic basis or metagenomics contribution from gut microbiome. Linking to diet effects will also teach about impact of diet on metabolic processes affected in disease. Collective information can then provide added tools for monitoring of disease, response to medications and also approaches for treatment of disease based on metabolic defects.

### 4. Precision Medicine Study

A study was conducted with the goal of enabling a precision medicine approach for wellness connecting digital recording and metabolic profiles for an individual.

The experiments were carried out on a 25-year-old healthy individual. The participant went through three different diet periods: a McDonald's diet (fast food), a Mediterranean diet, and a ketogenic (“Keto”) diet. Each of the diet periods was for two consecutive weeks and was followed by a one-week wash-out period before the next.

The meals for the McDonald's diet were bought from a McDonald's restaurant and included burgers, French fries, milkshakes, soft drinks (Coke), and coffee with coffee creamer or iced coffee. The participant consumed McDonald's for breakfast, lunch, and dinner and all snacks during the period were also bought from a McDonald's restaurant. The Mediterranean diet meals were prepared by the participant and consisted of Mediterranean diet-friendly ingredients such as fruits and vegetables, seafood, bread and pasta, and legumes. The Keto diet consumed was high in fat and protein and low in carbohydrates; the participant prepared the meals and used ingredients such as pork chops and chicken thigh (higher in fat levels), bacon, eggs, coffee with coconut oil, etc.

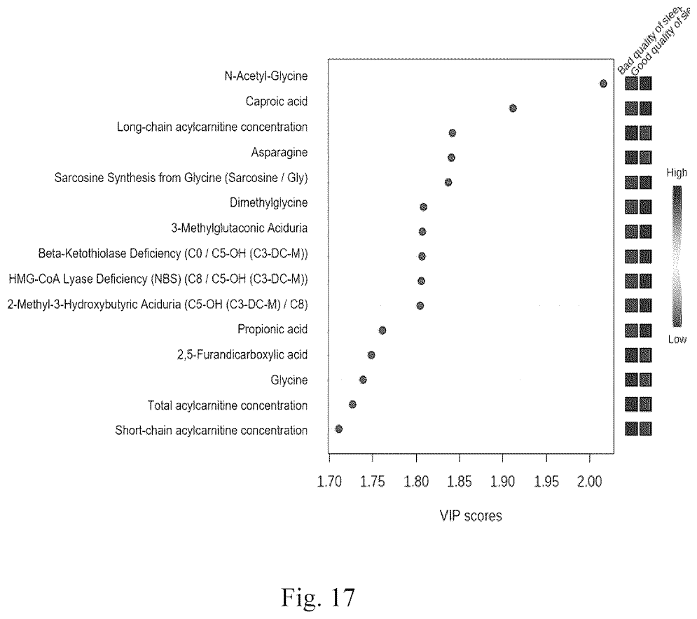

Blood pressure (BP) and Heart Rate: Blood pressure measurements were obtained using a smart, wearable BP monitor (YHE BP Doctor Pro Blood Pressure Smartwatch), throughout each day. The blood pressure was measured five times per day: after waking up, after each main meal (breakfast, lunch, and dinner), and before going to bed. The one-way analysis of variance (one-way ANOVA) performed on the blood pressure dataset indicates a significant difference between the diets with respect to the systolic (SYS) blood pressure measured after breakfast (p.value=0.0021171, f.value=7.2409). (FIG. 18).

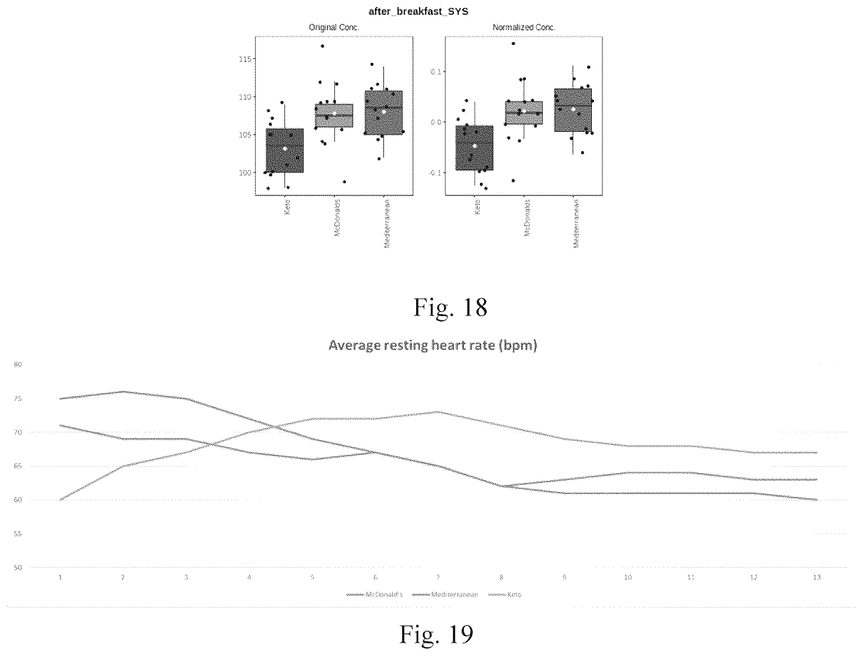

Heart rate was monitored every minute of each day using a Fitbit™ smartwatch. The Mediterranean diet had the greatest change over the course of the diet from day 1 to day 14, as it was lowered significantly and its overall value was 65.00±5.80. On the other hand, the Keto diet had the highest heart rate value amongst all the diets (mean+SD=68.92±2.28) and the McDonald's diet was average (mean+SD=65.5±2.64). Although the McDonald's diet and the Keto diet did not cause any significant increase or decrease in the heart rate, the daily heart rate averages were significantly decreased during the Mediterranean diet. (FIG. 19).

Body temperature: Body temperature was constantly monitored using a wearable body thermometer, the CORE sensor. The skin temperature was measured every five minutes throughout the day. The body temperature was slightly higher during the Keto diet (Mean±SD=37.06±0.09° C.). The temperature was relatively similar between the Mediterranean diet and the McDonald's diet (36.99±0.10° C. and 36.95±0.11° C., respectively).

Sleep, body temperature, breathing and heart rate: Sleep was monitored using the Fitbit™ smartwatch. The number of occurrences of each sleep stage, the number of minutes spent in each stage, the sleeping heart rate (bpm), tossing and turning (restlessness), the percentage of the sleeping heart rate measurements that was above the resting heart rate, and the temperature variance from baseline was measured using the Fitbit smartwatch and collected through the Fitbit API or the mobile phone application.

The participant reported having sleeping issues while being on the McDonald's diet. This was also confirmed through the collected data. The participant had the least amount of sleep (minutes) on the McDonald's diet (mean±SD=326.14±87.73), and the most amount of sleep while being on the Keto diet (mean±SD=435.38±65.92) while still feeling tired (along with headaches and significant mood changes) during the day. Sleep quality during the Mediterranean diet was average, and the length of time was in between the MD and KT diets with a mean value of 383.0±72.67. The number of minutes in the wake stage was positively correlated to the total amount of sleep, meaning the more minutes of sleep the participant had, the more minutes that were in the wake stage (McDonald's: 50.71±22.23, Mediterranean: 56.57±15.67, Keto: 78.0±28.73).

The number of occurrences of the deep sleep stage was the lowest on the McDonald's diet, and the highest on the Mediterranean diet (McDonald's: 2.64±1.00, Mediterranean: 3.21±1.25, Keto: 2.85±1.28). However, on average, the participant was in the deep sleep stage more while being on the McDonald's diet (78.36±25.66 minutes) in comparison to the Mediterranean diet (70.78±30.21), and the most while being on the Keto diet (84.53±20.57).

On average, the amount of time in the light sleep stage in the sleep cycles was positively correlated to the amount of sleep the participant had. With more total sleep time, there was more light sleep (occurrence and minutes). Likewise, for REM sleep, the minutes were also positively correlated to the amount of sleep in total.

The same pattern as the resting heart rate was observed in the sleeping heart rate. The Mediterranean diet was associated with the lowest heart rate and highest deviations from day 1 to day 14 (mean±SD=58.57±4.7), the McDonald's diet showed a more average heart rate (mean±SD=61.28±2.81), and the Keto diet showed the highest heart rate during sleep (mean±SD=64.54±2.81). For the Keto diet, 16.23±14.40% of the sleeping heart rate measurements were higher than the resting heart rate. This feature was lower during the McDonald's diet at 11.29±9.42% and was the lowest on the Mediterranean diet at 7.14±1.94%.

Restlessness was relatively similar between the McDonald's diet and the Mediterranean diet, and slightly higher during the Keto diet. The manufacturer of the smartwatch, Fitbit, does not disclose the precise formula for measuring restlessness, but it involves the sleeping heart rate, tossing and turning, and snoring, and then a number is assigned, with a higher number indicating more restlessness. The mean±SD values are as such: McDonald's=6.79±2.83, Mediterranean=6.07±1.38, Keto=7.00±2.35.

The highest number of deviations from the baseline temperature during sleep was observed during the McDonald's diet (−0.25±0.37° C.). The Keto diet had the least number of deviations from the baseline with −0.08±0.48° C., and for Mediterranean, it was −0.05=0.48° C.

Blood glucose: Blood glucose levels were continuously monitored using a Dexcom G6™ device. This device records the blood glucose value every 5 minutes via the sensor and transfers the collected data to the subject's phone through the transmitter. The collected data was accessed using the Dexcom G6 mobile application.

The one-way ANOVA analysis performed on the blood glucose datasets revealed a significant difference between the three diet groups (p.value: 4.80348E-09). On average, the McDonald's diet, with the highest level of blood glucose, had a mean of 6.34±0.21 mmol/L). On the other hand, the Keto diet with a mean and variation of 5.15±0.12 mmol/L. had the lowest levels of blood glucose among all diet groups. The Mediterranean diet was average, at 6.11±0.19 mmol/L.

Specific high-sugar foods, like Coke and milkshakes during the McDonald's diet or orange juice during the Mediterranean diet, caused the highest increases in the blood glucose level of the participant. The normal range of blood glucose level was considered to be between 3.9 and 10 mmol/L.

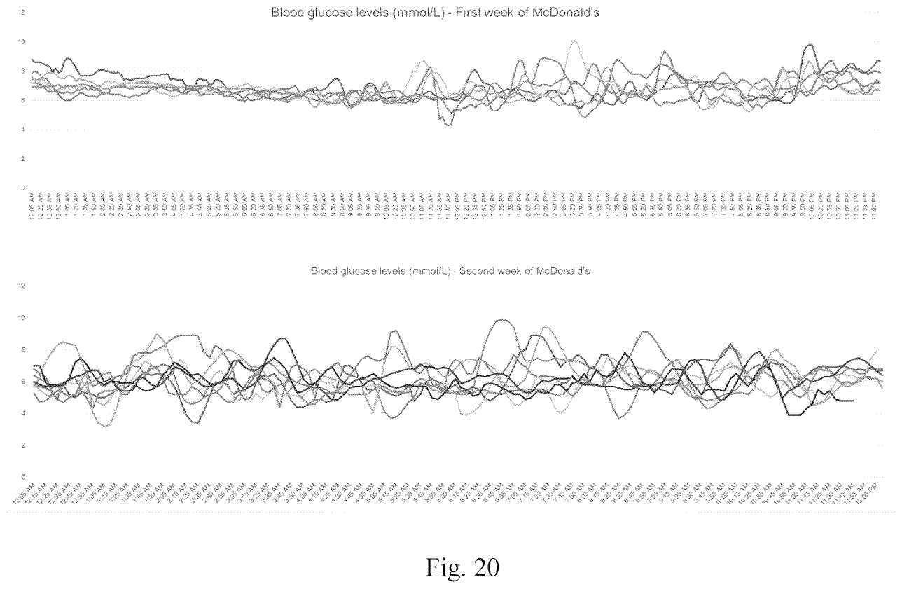

Observing the blood glucose level during the McDonald's diet reveals that during the second week of the diet, the intra-day variations increased, having higher increases or drops in the blood glucose level). As shown in the figures below, the variations in the blood glucose level during sleep were much higher in the second week of the McDonald's diet. However, in the first week of the diet, less variation was observed. (FIG. 20)

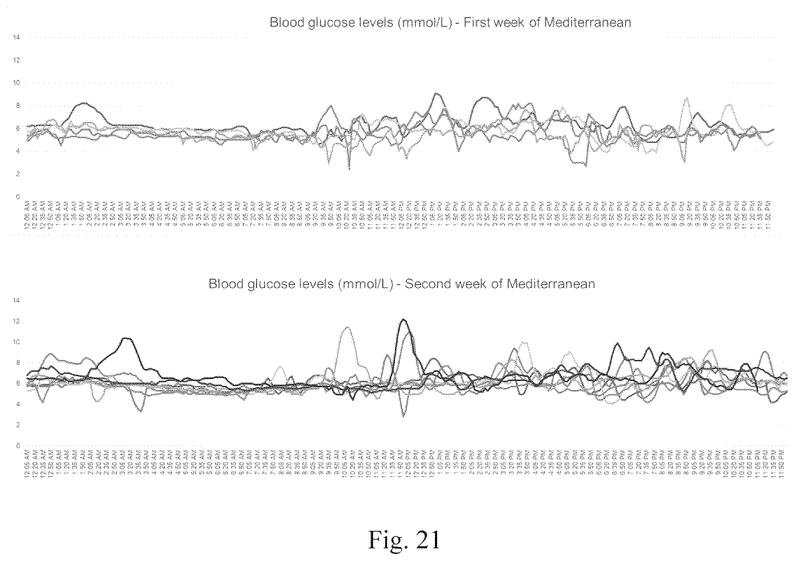

Comparing the first and second weeks of the Mediterranean diet shows a significant increasing shift in the blood glucose level. (FIG. 21)

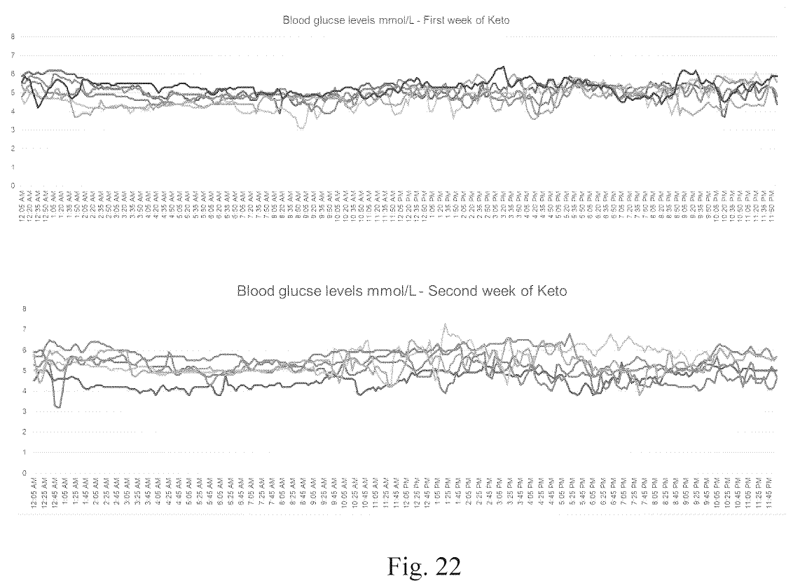

Blood glucose levels from the first and second weeks of the Keto diet are shown in FIG. 22.

Physical performance: Physical performance was monitored by performing the same amount of activity for 5 days/week during the diet periods. The physical activity consisted of walking on a treadmill for 40-45 minutes at between 5 km/h and 7 km/h. The participant followed a pattern of walking at a slower pace (5 km/h) for 3 minutes and then at a higher speed for 2 minutes (7 km/h), and repeating that until the activity time was over. Heart rate (during exercise) and recovery time (to resting heart rate after exercise) were used to assess performance and fitness.

Looking at the heart rate values during the exercises showed that the average exercise heart rate values (bpm) during the McDonald's diet was the lowest with a mean±SD of 125.76±20.68 bpm. The Mediterranean diet with a mean and standard deviation of 130.34±21.16 bpm and the Keto diet with a mean and standard deviation of 131.70±22.54 bpm showed higher heart rate values. Considering that the exercise time was similar between all days, the Mediterranean diet had the lowest average time in the cardio level (13.2 minutes) and the most time in fat burn (19.4 minutes) between all 3 diets. During the Keto diet, while during the same exercise, the participant's heart rate on average reached the cardio level for 15.8 minutes and the fat burn level for 15.4 minutes. These numbers were higher during the McDonald's diet with 16.5 minutes in the cardio stage and 22. 1 minutes in the fat burn stage. On average, while the participant was on the McDonald's diet, it took 5.67 minutes to come back to the resting heart rate. This value was slightly lower during the Keto diet (5.5 minutes) and the lowest on the Mediterranean diet (5 minutes).

Body temperature did not vary significantly during the exercises between the diets. The Mediterranean diet with a mean and SD of 37.36±0.24° C. had the lowest temperature during the exercises. McDonald's and the Keto diet with a mean and SD of 37.40±0.30° and 37.49±0.11° C., respectively, had a higher temperature. Similarly, with respect to the daily blood glucose levels, the Keto diet had a mean SD of 5.07±0.47 mmol/L and was the lowest among all the diets. The McDonald's diet and the Mediterranean diet, with values of 5.75±0.49 mmol/L and 5.79±0.48 mmol/L respectively, had higher levels of blood glucose during the exercises.

Mental performance: Mental performance was monitored by performing multiple mental capability assessments each day at approximately the same time of day (e.g., 11 am in the morning). The tests were done using a laptop computer with a simple computer testing interface. Each of the mental performance tests were programmed using the Python programming language. The tests included a reaction time test, a trail making test, a digit span test, a serial subtraction test, and the Stroop effect test. Scores for each test were recorded by the computer. Each test was randomized so that the subject could not improve their performance through learning or via repetition.

The overall results of the mental cognition of the participant were higher during the Mediterranean and the Keto diets, and the worst scores were obtained during the McDonald's diet, which could be due to sleep deficiency that was experienced at that time.

Results: The one-way ANOVA analysis of significance performed on the mental capability assessment results showed a significant difference between the diets in the digit span test (p.value=1.4342E-8), reaction time test (p.value=6.6508E-4), and the trail-making test (p.value=0.0012958).

The performance of the participant on the digit span test (the higher the number the better) while on the McDonald's diet had the lowest score and variation (mean±SD=63.92±7.60). The Keto diet had the most variation throughout the diet period (mean±SD=83.093±11.95). The results during the Mediterranean diet (with a mean value of 87.13) were the closest to those during the regular diet (mean=89.995).

Reaction time test—The Mediterranean diet had the highest variation and mean value in comparison to the other diets (Mean±SD=0.6907±0.11). However, the McDonald's and Keto diets (mean±SD=0.5722±0.11, and mean±SD=0.6236±0.07, respectively) remained fairly similar in comparison to the regular diet.

The time it took the participant to finish the trail-making test was also different between the diets. In general, the participant was slowest in making the traces while on the McDonald's diet (mean±SD=36.0714±6.93 seconds). The participant was fastest during the Mediterranean diet (28.2857±4.09 seconds), while the times during the Keto diet were in between (33.0714±5.30 seconds).

Other mental performance tests conducted throughout the study did not show any significance.

Mood: Mood and emotional state were monitored and tracked every three days by answering a detailed questionnaire called the Profile of Mood State. The subject recorded their moods and feelings on day 1, day 4, day 7, day 10 and day 14 of each diet. Daily tracking of mood and emotional state was also done by taking detailed notes entered into a daily diary throughout the diet periods.

The participant kept a detailed diary of mood changes and a daily journal during the diet periods. Using the Profile of Mood State standard form, the moods and feelings were transformed into a quantitative value to facilitate the process of data analysis and comparison. The one-way ANOVA test results showed significance in all categories:

| Categories | f. value | p. value | −LOG10 | FDR |
|---|---|---|---|---|
| Confusion | 62.727 | 4.43E−07 | 6.3538 | 3.10E−06 |
| Tension | 31.62 | 1.65E−05 | 4.7836 | 5.76E−05 |
| Fatigue | 20.361 | 0.0001391 | 3.8568 | 0.000324 |
| Anger | 17.234 | 0.0002966 | 3.5278 | 0.000519 |
| Total mood | 14.508 | 0.0006271 | 3.2027 | 0.000732 |
| disturbance | | | | |
| Depression | 14.505 | 0.0006277 | 3.2022 | 0.000732 |
| Vigour | 7.4497 | 0.007882 | 2.1034 | 0.007882 |

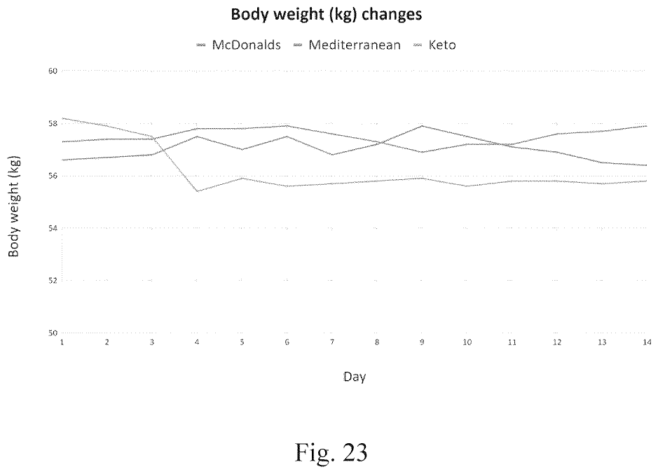

Body weight (kg): General body measurements of weight, hip, waist, and neck circumferences were conducted daily. The measured values were used to calculate body fat percentage (based on the U.S. Navy method) and body mass index (BMI kg/m²). The one-way ANOVA analysis of the general body information (body weight, BMI, body fat) showed significant changes in body weight (p. value=7.35E-6) and BMI (p.value=1.9693E-6) between the diet, however, no significant changes were observed in body fat composition. In general, body weight had limited fluctuations during the MD (mean±SD=57.50±0.30), MT (mean±SD=57.03±0.45), and the regular (mean±SD=57.30±0.23) diets. However, there was a significant drop in body weight (2.8 kg) observed within the first few days of the KT diet (mean±SD=56.19±0.93), and the weight was maintained until the last day of the diet. The lost weight was regained after 2-3 weeks of finishing the diet. (FIG. 23)

Microbiome: Microbial diversity and microbial “microbotypes” were measured by performing a gut microbiome test as conducted by (EasyDNA). The tests were conducted after finishing each diet period to have a better understanding of the changes in the gut microbiome that were affected by the diet.

The 23andme genetics test reported that variants were detected in the results, except that one variant of age-related macular degeneration detected in the participant's genetic makeup indicated that the participant is not likely at high risk.

The gut microbiome test results for the baseline period showed that the overall diversity of the microbiome, the abundance of vitamin-producing bacteria, fibre-degrading bacteria, and probiotic bacteria were within the optimal range. Also, the participant was receiving the optimal amount of protein and fat from her regular diet. The microbiome slightly favoured obesity and inflammatory condition of the gut, and no pathogenic bacteria were detected in the microbiome. The tests done after the McDonald's and the Mediterranean diets showed the same pattern. These results were altered after finishing the Keto diet. The microbiome was not in favour of obesity or inflammatory condition of the gut with the Keto diet. However, there was an increase in the abundance of pathogenic bacteria and the abundance of probiotic bacteria in the microbiome was slightly low.

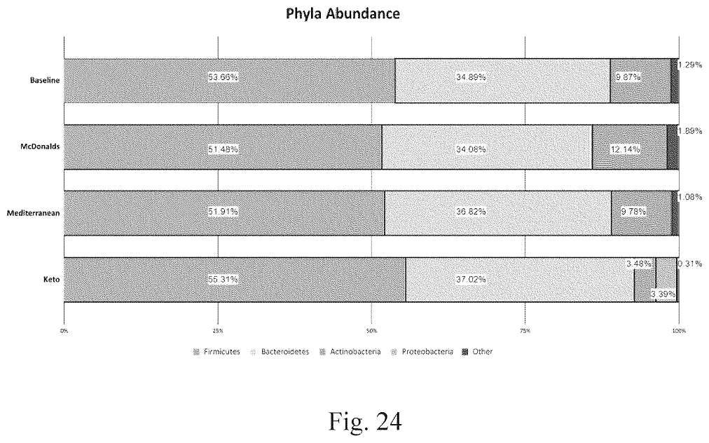

The most abundant phyla in all the microbiome test results were Firmicutes, Bacteroidetes, and Actinobacteria. However, the amount of Actinobacteria was slightly decreased after the Keto diet, and 3.39% of Proteobacteria was detected in the microbiome which was absent in the other test results. There was also a negligible amount of less than 2% of all other phyla detected. (FIG. 24)

The genus Prevotella, which comprised 25.61% of the participant's microbiome for the baseline, remained fairly constant after the McDonald's diet (24.5%), and reached a maximum value of 29.77% after the Mediterranean diet. It was generally the most abundant genus, except that it decreased after the keto diet to 15.28%. The same decreasing pattern was observed with the genus Bifidobacterium, which remained approximately constant throughout the first two diets (baseline value: 4.91%, McDonald's diet: 5.74%, Mediterranean diet: 6.75%), but drastically dropped to 0.06% after the Keto diet. The genus Bacteroides had a value of 7.45% at baseline, a similar value of 7.42% after the McDonald's, a slightly lower value of 5.87% after the Mediterranean diet, but was significantly increased to 19.26% after the Keto diet. It is the second most abundant genus in all the diets, except for the Keto diet in which it was the most abundant and Prevotella was second most abundant. The amount of the genus Lactobacillus, which was 0.52% of the microbiome at baseline, was substantially decreased after the diets to 0.13% after the McDonald's diet, 0.17% after the Mediterranean diet, and 0.05% after the Keto diet. Akkermansia municiphila, which was 0% of the microbiome (or otherwise undetectable) at the baseline level and after the McDonald's diet, was increased to 0.004% after the Mediterranean diet and 0.02% after the Keto diet.

Faecalibacterium, as one of the most abundant genera at baseline (with 5.8% abundance), stayed fairly the same after the McDonald's and Mediterranean diets, but it increased to 7.7% after the Keto diet. Holdemanella at 5.76% at baseline, increased to 6.92% after the McDonald's diet and dropped down to 3.42% after the Mediterranean diet. The abundance of this genus was close to the baseline value after the Keto diet (6.12%).

The abundance of Ruminococcus was close to the baseline value after the Keto diet (4.17% and 3.79%, respectively), but it greatly decreased to 1.08% and 1.81% after the McDonald's and the Mediterranean diets, respectively. The percentage of Blautia detected in the microbiome was 3.99% at the baseline level. This value decreased to 2.63% after the McDonald's diet and to 3.12% after the Keto diet, but it reached 5.73% after the Mediterranean diet. The abundance of Collinsella and Romboutsia, at 3.92% and 3.29%, respectively, at the baseline level, increased to 5.54% and 4.97% after the McDonald's diet. However, the abundance of both genera decreased to 2.25% and 2.14% after the Mediterranean diet, and to 2.28% and 1.05% after the Keto diet, respectively. Coprococcus with 2.97% at the baseline level, decreased to 1.33% after the McDonald's diet, but it increased to 3.75% after the Mediterranean diet and to 3.85% after the Keto diet.

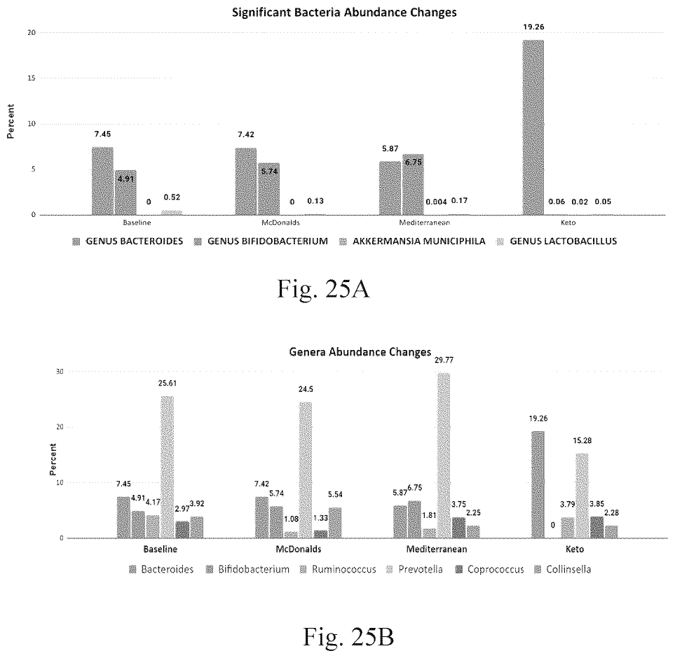

Data is shown in FIGS. 25A and 25B.

Plasma Collection: The subject collected blood and urine samples. Blood samples were obtained on a daily basis before breakfast (fasting blood) using a TASSO+ blood collection device. After collection (about 2 mL of blood), the samples were centrifuged in order to separate the plasma, and frozen at −20° C. until transferred to a −80° C. freezer at the University of Alberta for longer term storage.

LC-MS Assay: Metabolomic analyses were performed on the collected plasma samples using an LC-MS/MS assay developed by The Metabolomics Innovation Centre (TMIC), called the TMIC MEGA assay. This assay is able to perform targeted, absolutely quantitative metabolic profiling of up to 645 metabolites including an additional 200 metabolite ratios and sums. For the serum samples analyzed here, we obtained quantitative results for an average of 636 metabolites (including biogenic amines, amino acids and amino acid-related metabolites, phosphatidylcholines (PCs), lysophosphatidylcholines (LysoPCs), sphingomyelins (SMS) and hydroxysphingomyelins (SM(OH)s), acylcarnitines (ACs), triglycerides (TGs), diglycerides (DGs), organic acids, etc.). The serum samples also yielded 188 combinations of metabolite sums and ratios—giving a total of 824 absolutely quantitative metabolite values.

The TMIC MEGA is a triple quadrupole MS-based assay that uses chemical derivatization, analyte extraction and separation, combined with selective mass-spectrometric detection using multiple reaction monitoring (MRM) to identify and quantify metabolites. The assay uses a combination of direct injection (DI) and reverse-phase ultrahigh-performance liquid chromatography (UHPLC). Isotope-labeled internal standards (ISTDs) along with other ISTDs are used for accurate metabolite quantification. To support high throughput analysis, the assay uses a multi-sample plate assay. The assay also requires a specific set of standard reagents and solvents. Stock solutions of each standard used in the assay are prepared by dissolving accurately weighed solids in double-distilled water (ddH₂O). Calibration curve standards are obtained by mixing and diluting each of the corresponding stock solutions with ddH₂O. For amino acids, biogenic amines, carbohydrates, acylcarnitines and derivatives, as well as all lipids and their derivatives, stock solutions of isotope-labelled compounds were also prepared in the same way. A working internal standard (ISTD) solution mixture in ddH₂O was also made by mixing all the prepared isotope-labeled stock solutions together. For organic acids, stock solutions of isotope-labelled compounds were prepared by dissolving the accurately weighed solids in 75% aqueous methanol. A working internal standard solution mixture in 75% aqueous methanol was made by mixing and diluting all the isotope-labelled stock solutions.

The TMIC MEGA assay employs a 96 deep-well plate with a separate filter plate attached via sealing tape. The first 14 wells are used for a blank sample, three zero-point samples, seven standard-containing or calibration samples and three quality control (QC) samples. Prior to running the plasma samples, vials containing the plasma were removed from the freezer, thawed on ice and were vortexed and centrifuged at 13,000× g. 10 μL of each plasma sample was loaded onto the center of the filter on the upper 96-well plate and dried in a stream of nitrogen. Subsequently, phenyl-isothiocyanate (PITC) was added for the derivatization of all amine-containing metabolites. After incubation, the filter spots were dried again using an evaporator. Extraction of the metabolites was then achieved by adding an ammonium acetate/methanol mixture (5 mM ammonium acetate dissolved in 300 μL methanol). Then the extracts were centrifuged into the lower 96-deep well plate, and then diluted with the MS running solvent. For organic acid analysis, 150 μL of ice-cold methanol and 10 μL of an isotopically-labeled ISTD mixture was added to 50 μL of serum sample for overnight protein precipitation. Then the sample was centrifuged at 13,000× g for 20 min at 4° C. 50 μL of the supernatant was loaded onto the center of a selected well of the 96-deep well plate, followed by the addition of 3-nitrophenylhydrazine (3-NPH), which serves as an organic-acid specific derivatization reagent. After incubation for 2 h, butylated hydroxytoluene (BHT), which is used as a stabilizer, and water were added prior to LC-MS injection. All chemically-derivatized samples were delivered to an Exion Qtrap® 5500 tandem mass spectrometer (Applied Biosystems/MDS Analytical Technologies, Foster City, CA) equipped with an Agilent 1290 series UHPLC system (Agilent Technologies, Palo Alto, CA) with an Agilent Zorbax C18 column. Samples were also delivered to the mass spectrometer via direct injection (DI) to detect and quantify lipids and acylcarnitines. Data analysis was done using Analyst 1.6.2.

### Results

The different diets served as external perturbations which led to extended periods of enhanced or diminished physical performance, mental performance, diminished/elevated blood pressure, diminished/elevated heart rate, sleep quality, high/low weight, different microbotypes, etc. This allowed specific groupings of similar digital health/performance readouts for 3+ consecutive days to be obtained. For example, heart rate (bpm), over the 6 weeks of diet testing, led to multiple, consecutive days with similar heart rate values that could be clearly classified as high, average and low heart rate groups. Using these “digital health status groupings” or DHSGs, it was then possible to compare the plasma metabolomic profiles to those DHSGs and to identify metabolites that are associated or are potentially causal for changed DHSG ratings. In some embodiments, a DHSG is an example of a health stratification group.

For instance, high heart rates with this subject are associated with high levels of branched chain amino acids (BCAA), 2-Hydroxy-3-methylvaleric acid, and short chain acylcarnitines. A total of 12 DHSGs were identified and typically classified into 2 or 3 categories (high/low, or high/medium/low or good/bad). Some digitally derived features, such as sleep, were more complex and consisted of multiple features (the number of times each sleep stage occurred, the number of minutes in each stage throughout one night's sleep, the sleeping heart rate, restlessness, etc.). These were separated into bins of good, bad, and average sleep quality based on the ranges of the collected data. Since the features do not all affect the sleep quality equally, a weighted scoring system was applied to help better understand the sleep data. Plasma metabolomics data for all identified DHSGs (sleep, heart rate, weight, etc.) were then analyzed using MetaboAnalyst 5.0, a popular metabolomics data analysis tool. (http://www.metaboanalyst.ca/).

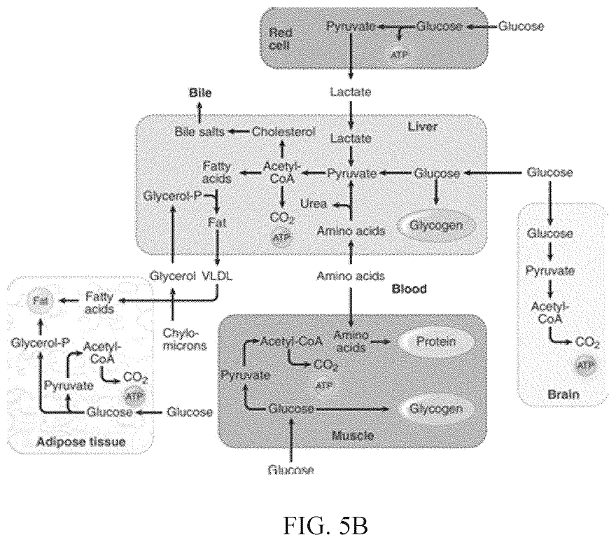

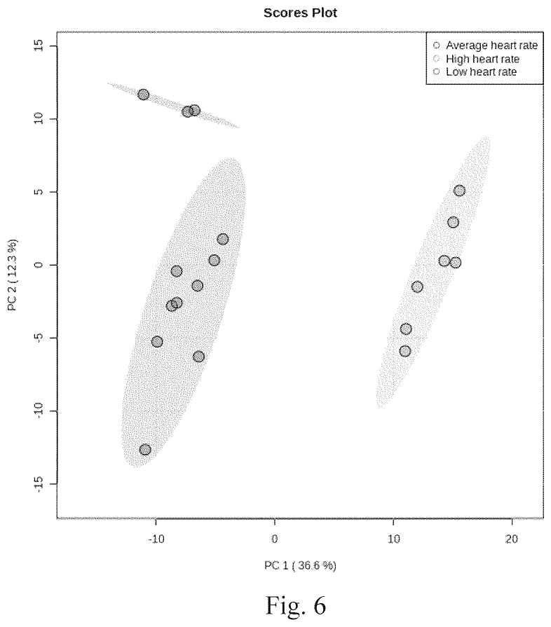

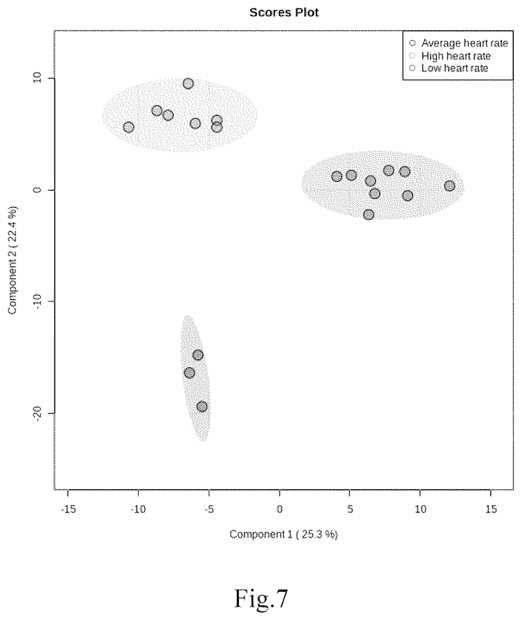

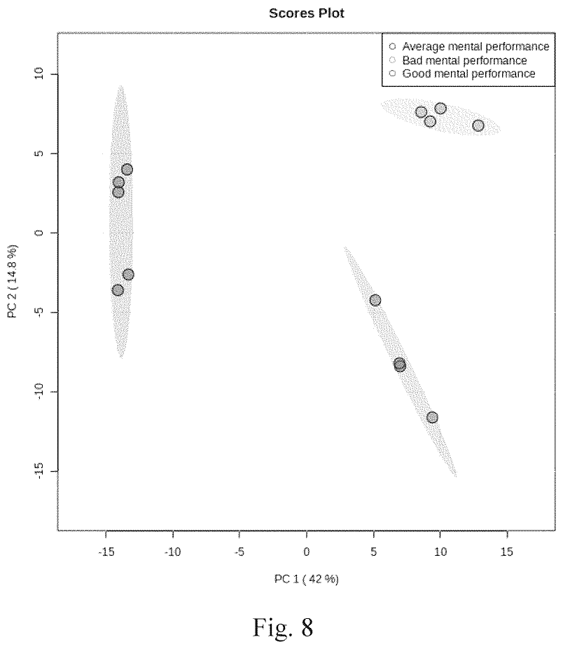

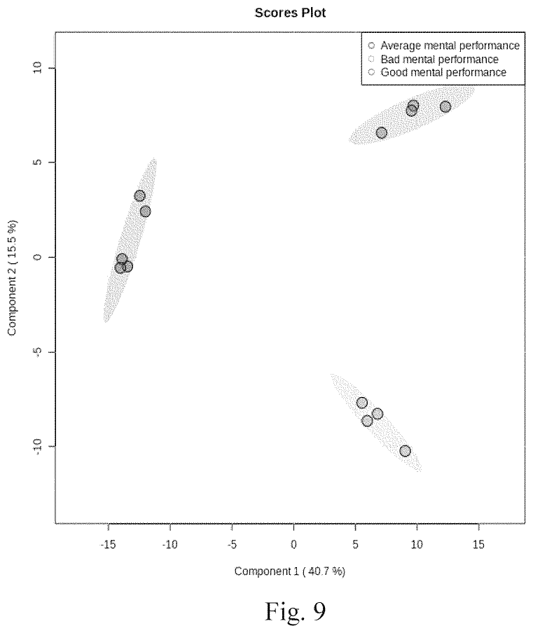

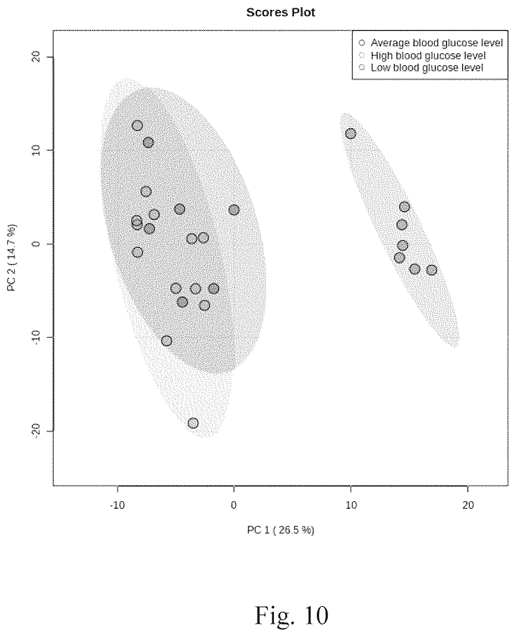

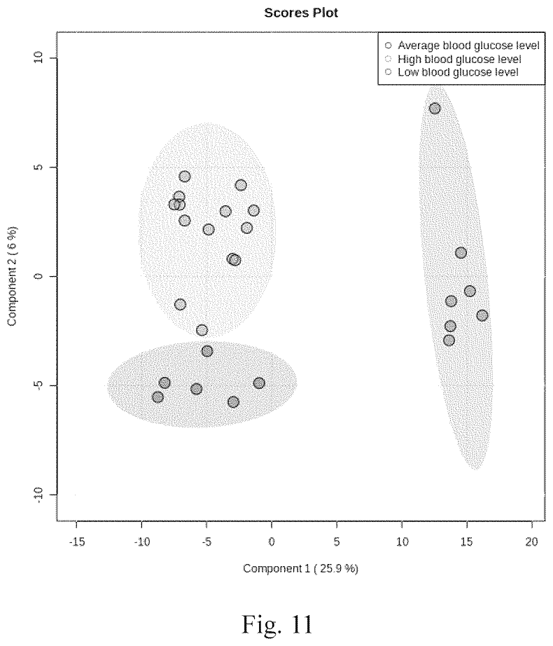

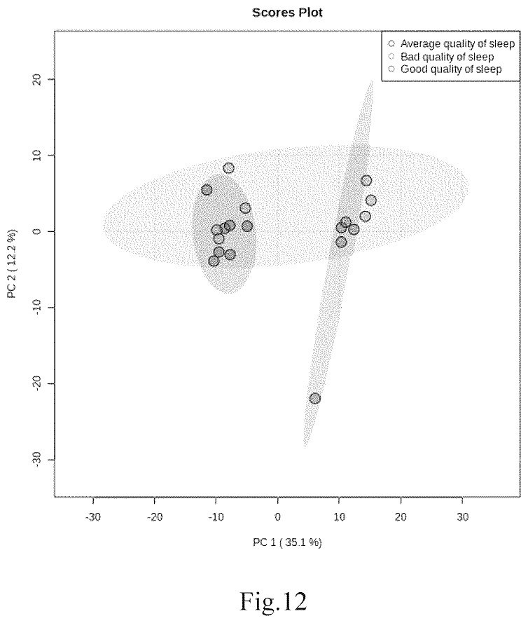

As seen in FIGS. 6-13, Principle Component Analysis (PCA), and Partial Least Squares Discriminant Analysis (PLS-DA) on the plasma metabolomics data revealed that the plasma metabolome composition of an individual can provide a clear indication of their individual physiological profile or DHSG. Both the PCA scores plots and the PLS-DA plots of the illustrated DHSGs indicate clear associations of certain metabolites and metabolomic profiles with specific physiological states or specific DHSGs. The clearly separated clusters of similar physiological attributes (regardless of the diet and lifestyle interventions of the subject) supports this idea.

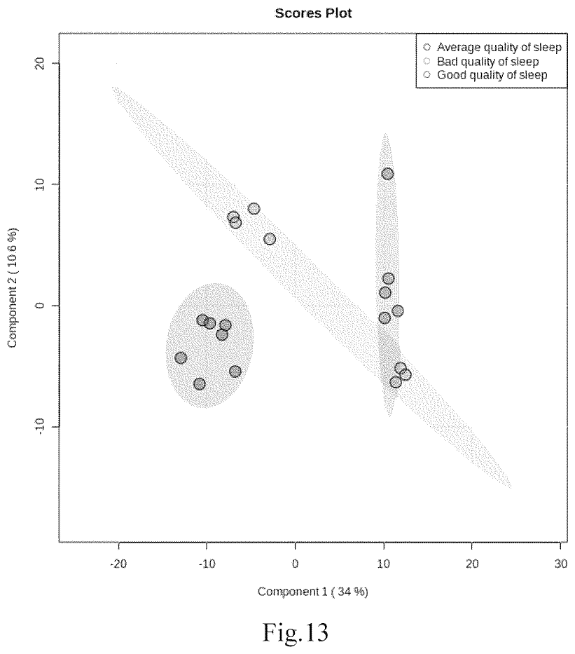

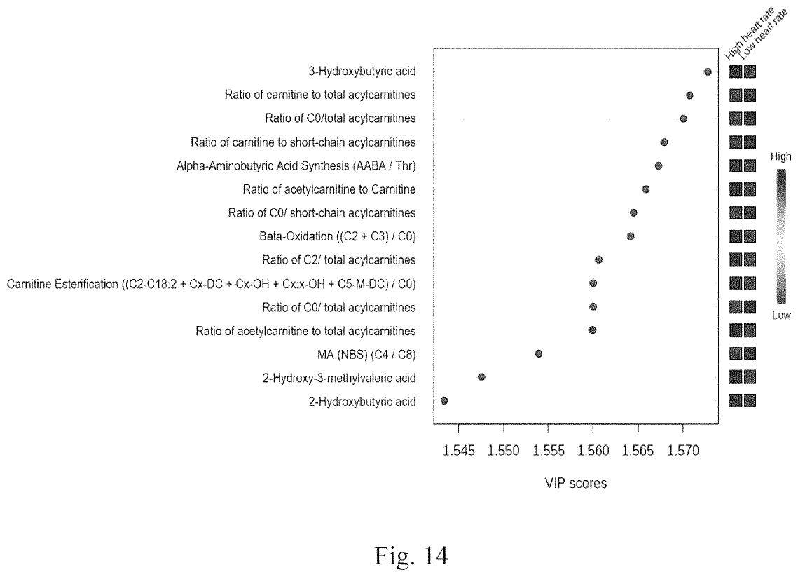

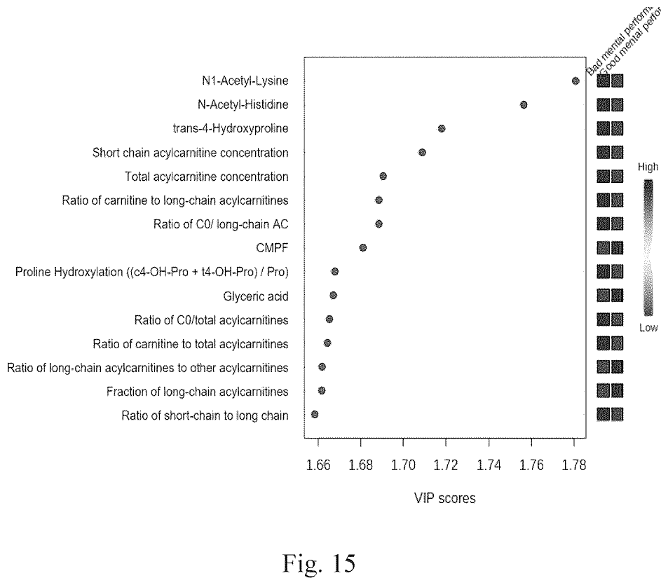

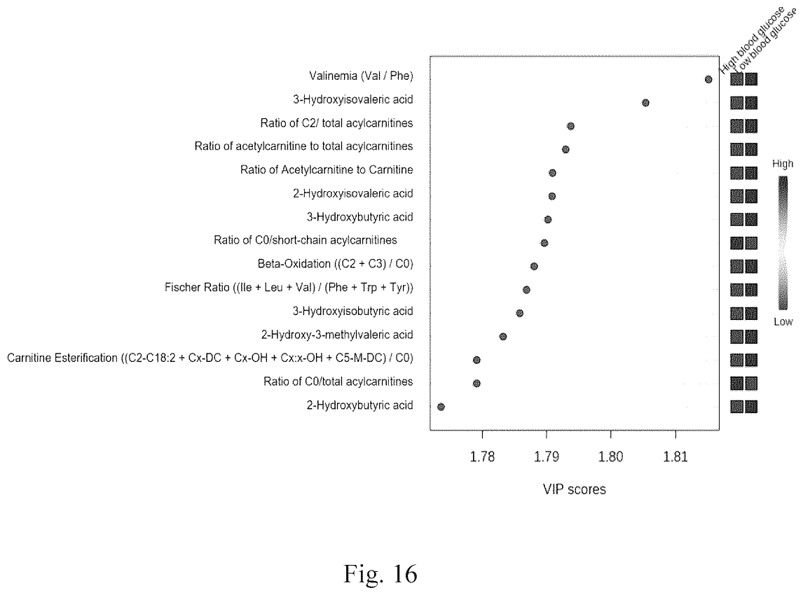

FIGS. 14-17 show the VIP plots of the most important metabolites and metabolite ratios that are associated with these different DHSGs.

In one particular example, as an example of demonstrating how to connect the metabolic profile to the digital output, a participant had the lowest free carnitine (C0) to total acylcarnitine (total AC) concentration ratio during the Keto diet, with a mean value of 1.25±1.11 for the entire 14 days and 0.86±0.21 for days 3-14 (days 1 and 2 being higher after coming off her regular diet in the washout period). During the Keto diet, the participant also had the most sleep minutes (at a mean of 435±66 minutes per night as reported by the Fitbit smartwatch) yet was tired during the day and had headaches. It is recognized that a C0/total AC ratio below 2.5 is indicative of mitochondrial dysfunction. (The participant also reported being moody and it is also known that the acylcarnitine profile affects the central nervous system.) Therefore, for this participant, it can be inferred that the Keto diet caused physiological changes involving mitochondrial dysfunction, leading to less energy being produced and thus requiring the participant to sleep more.

One skilled in the art will readily appreciate that the present disclosure is well adapted to carry out the objects and obtain the ends and advantages mentioned, as well as those inherent therein. The present disclosure described herein are presently representative of preferred embodiments, are exemplary, and are not intended as limitations on the scope of the present disclosure. Changes therein and other uses will occur to those skilled in the art which are encompassed within the spirit of the present disclosure as defined by the scope of the claims.

No admission is made that any reference, including any non-patent or patent document cited in this specification, constitutes prior art. In particular, it will be understood that, unless otherwise stated, reference to any document herein does not constitute an admission that any of these documents forms part of the common general knowledge in the art in the United States or in any other country. Any discussion of the references states what their authors assert, and the applicant reserves the right to challenge the accuracy and pertinence of any of the documents cited herein. All references cited herein are fully incorporated by reference, unless explicitly indicated otherwise. The present disclosure shall control in the event there are any disparities between any definitions and/or description found in the cited references.

1. A method of improving the health or wellness of a subject, using a master database (MetabosensorDB) created by collecting continuous digital health data and metabolomic data for a population of individuals over an extended time period where at least one perturbation is applied, and where standard multivariate statistical methods are used to group members of the population into health stratification groups, comprising the steps of:
(a) Collecting digital health and metabolic data for a subject over a shorter time period;
(b) Comparing the subject's digital health phenotype (DHP) and metabolic health phenotype (MHP) to see which health stratification group in the MetaboSensorDB the subject matches with;
(c) apply a perturbation to the subject, which perturbation is associated with a desired health or wellness outcome for individuals in the health stratification group matched by the subject.

2. The method of claim 1 wherein the at least one perturbation comprises one or a combination of a diet, an exercise, use of a supplement, probiotic, nutraceutical and/or a medication, a clinical treatment or therapy, a prophylactic treatment or therapy.

3. The method of claim 1, wherein the subject continues to collect digital health data and/or metabolomic data and determine the effect of the perturbation on the DHP and/or MHP of the subject, which data and effect is added to the MetabosensorDB.

4. The method of claim 1, wherein if the subject experiences a change in DHP and/or MHP which is different than the matched health stratification group, the subject is moved to another health stratification group, or a subgroup may be identified.

5. The method of claim 1, wherein the MetaboSensorDB further comprises one or both of genomic profile data and microbiome profile data from the population of individuals, and the subject collects and compares such genomic profile data and/or microbiome profile data in the matching step to find a matching health stratification group.

6. The method of claim 1, wherein the shorter time period is a single 24 hour period.

7. The method of claim 1, wherein the shorter period is more or less than 24 hours.

8. A system for improving the health or wellness of a subject; comprising:
(a) a master database (MetabosensorDB) comprising digital health data and metabolomic data collected for a population of individuals over an extended time period where at least one perturbation is applied, and where standard multivariate statistical methods are used to group members of the population into a plurality of health stratification groups;
(b) at least one device for collecting digital health data from a subject;
(c) a metabolomic profile derived from a biological sample from the subject;
(d) a computer operatively connected to the at least one device and configured to:
i. receive the subject digital health data and metabolomic profile, and compare to the plurality of health stratification groups to match the subject to a health stratification group; and
ii. identify at least one perturbation which is predicted to improve the subject's health or wellness; and
iii. communicate the predicted at least one perturbation to the subject.

9. The system of claim 8 wherein the at least one perturbation comprises one or a combination of a diet, an exercise, use of a supplement, probiotic, nutraceutical and/or a medication, a clinical treatment or therapy, a prophylactic treatment or therapy.

10. The system of claim 8, wherein the computer is further configured to move the subject to another health stratification group, or a subgroup, if the subject experiences a change in DHP and/or MHP which is different than the matched health stratification group.

11. The system of claim 8, wherein the MetaboSensorDB further comprises one or both of genomic profile data and microbiome profile data from the population of individuals, and the computer is configured to receive and compares such genomic profile data and/or microbiome profile data in the matching step to find a matching health stratification group.
